SEQUENCE LISTING (70 sequences)

SEQ ID NO: 1 - protein, 9 aa, synthetic construct
GGSISNSYY

SEQ ID NO: 2 - protein, 7 aa, synthetic construct
IYHSGNT

SEQ ID NO: 3 - protein, 14 aa, synthetic construct
VTQDGVGATTTEEY

SEQ ID NO: 4 - protein, 14 aa, synthetic construct
TSQDGVGATTTEEY

SEQ ID NO: 5 - protein, 14 aa, synthetic construct
TTQNGVGATTTEEY

SEQ ID NO: 6 - protein, 14 aa, synthetic construct
ATQNGVGATTTEEY

SEQ ID NO: 7 - protein, 7 aa, synthetic construct
IYYSGST

SEQ ID NO: 8 - protein, 14 aa, synthetic construct
ATQDGVGATTTEEY

SEQ ID NO: 9 - protein, 9 aa, synthetic construct
GGSISSSYY

SEQ ID NO: 10 - protein, 7 aa, synthetic construct
IYYSGSI

SEQ ID NO: 11 - protein, 121 aa, synthetic construct
QLQLQESGPGLVKPSETLSLTCTVSGGSISNSYYWGWIRQPPGKGLEWIGSIYHSGNTYYNPSLKSRVTISVDTSKNQFSLKLSSVTAADTAVYYCVTQDGVGATTTEEYWGQGTLVTVSS

SEQ ID NO: 12 - protein, 121 aa, synthetic construct
QLQLQESGPGLVKPSETLSLTCTVSGGSISNSYYWGWIRQPPGKGLEWIGSIYHSGNTYYNPSLKSRVTMSVDTSKNQFSLKLSSVTAADTAVYYCTSQDGVGATTTEEYWGQGTLVTVSS

SEQ ID NO: 13 - protein, 121 aa, synthetic construct
QLQLQESGPGLVKPSETLSLTCTVSGGSISNSYYWGWIRQPPGKGLEWIGSIYHSGNTYYNPSLKSRVTISVDTSKNQFSLKLSSVTAADTAVYYCTTQNGVGATTTEEYWGQGTLVTVSS

SEQ ID NO: 14 - protein, 121 aa, synthetic construct
QLQLQESGPGLVKPSETLSLTCTVSGGSISNSYYWGWIRQPPGKGLEWIGSIYHSGNTYYNPSLKSRVTISVDTSKNQFSLKLSSVTAADTAVYYCTSQDGVGATTTEEYWGQGTLVTVSS

SEQ ID NO: 15 - protein, 121 aa, synthetic construct
QLQLQESGPGLVKPSETLSLTCTVSGGSISNSYYWGWIRQPPGKGLEWIGSIYHSGNTYYNPSLKSRVTISVDTSKNQFSLKLSSVTAADTAVYYCATQNGVGATTTEEYWGQGTLVTVSS

SEQ ID NO: 16 - protein, 121 aa, synthetic construct
QLQLQESGPGLVKPSETLSLTCTVSGGSISNSYYWGWIRQPPGKGLEWIGSIYYSGSTYYNPSLKSRVTISVDTSKNQFSLSLNSVTAADTAVYYCATQDGVGATTTEEYWGQGTLVTVSS

SEQ ID NO: 17 - protein, 121 aa, synthetic construct
QLQLQESGPGLVKPSETLSLTCTVSGGSISSSYYWGWIRQPPGKGLEWIGSIYHSGNTYYNPSLKSRVTISVDTSKNQFSLKLSSVTAADTAVYYCATQNGVGATTTEEYWGQGTLVTVSS

SEQ ID NO: 18 - protein, 121 aa, synthetic construct
QLQLQESGPGLVKPSETLSLTCTVSGGSISSSYYWGWIRQPPGKGLEWIGSIYHSGNTYYNPSLKSRVTISVDTSKNQFSLKLSSVTAADTAVYYCTSQDGVGATTTEEYWGQGTLVTVSS

SEQ ID NO: 19 - protein, 121 aa, synthetic construct
QLQLQESGPGLVKPSETLSLTCTVSGGSISNSYYWGWIRQPPGKGLEWIGSIYYSGSIHYNPSLKSRVTISVDTSKNQFSLKLSSVTAADTAVYYCTSQDGVGATTTEEYWGQGTLVTVSS

SEQ ID NO: 20 - protein, 8 aa, synthetic construct
GFTFDDYA

SEQ ID NO: 21 - protein, 8 aa, synthetic construct
GFTFANYA

SEQ ID NO: 22 - protein, 8 aa, synthetic construct
GFTFNNYA

SEQ ID NO: 23 - protein, 8 aa, synthetic construct
GFTFADYA

SEQ ID NO: 24 - protein, 8 aa, synthetic construct
GFTFDNYA

SEQ ID NO: 25 - protein, 8 aa, synthetic construct
GFTFHNYA

SEQ ID NO: 26 - protein, 8 aa, synthetic construct
ISWNSGSI

SEQ ID NO: 27 - protein, 16 aa, synthetic construct
AKDSRGYGDYRLGGAY

SEQ ID NO: 28 - protein, 16 aa, synthetic construct
AKDSRGYGDYSRGGAY

SEQ ID NO: 29 - protein, 16 aa, synthetic construct
AKDSRGYGDYSLGGAY

SEQ ID NO: 30 - protein, 16 aa, synthetic construct
AKDSRGYGSYSRGGAY

SEQ ID NO: 31 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 32 - protein, 123 aa, synthetic construct
EVQLVESGGGLVKPGGSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 33 - protein, 123 aa, synthetic construct
EVQLVESGGGLVQPGRSLRLSCAASGFTFDDYAMHWVRQAPGKGLEWVSGISWNSGSIGYADSVKGRFTISRDNAKKSLYLQMNSLRAEDTALYYCAKDSRGYGDYSRGGAYWGQGTLVTVSS

SEQ ID NO: 34 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGDYSLGGAYWGQGTLVTVSS

SEQ ID NO: 35 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 36 - protein, 123 aa, synthetic construct
EVQLVESGGGLVQPGRSLRLSCAASGFTFANYAMHWVRQAPGKGLEWVSGISWNSGSIDYADSVKGRFTISRDNAKNSLYLQMNSLRAEDTALYYCAKDSRGYGDYSRGGAYWGQGTLVTVSS

SEQ ID NO: 37 - protein, 123 aa, synthetic construct
EVQLVESGGGLVQPGRSLRLSCAASGFTFNNYAMHWVRQAPGKGLEWVSGISWNSGSIGYADSVKGRFTISRDNAKNSLYLQMNSLRGEDTALYYCAKDSRGYGDYSRGGAYWGQGTLVTVSS

SEQ ID NO: 38 - protein, 123 aa, synthetic construct
EVQLVESGGGLVQPGGSLRLSCAASGFTFADYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 39 - protein, 123 aa, synthetic construct
EVQLVESGGGLVQPGRSLRLSCAASGFTFDNYAMHWVRQAPGKGLEWVSGISWNSGSIGYADSVKGRFTISRDNAKKSLYLQMNSLRAEDTALYYCAKDSRGYGDYSRGGAYWGQGTLVTVSS

SEQ ID NO: 40 - protein, 123 aa, synthetic construct
EVQLVESGGGLVQPGRSLRLSCAASGFTFDNYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 41 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 42 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGSYSRGGAYWGQGTLVTVSS

SEQ ID NO: 43 - protein, 123 aa, synthetic construct
EVQLVESGGGLVQPGRSLRLSCAASGFTFHNYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 44 - protein, 123 aa, synthetic construct
EVQLVESGGGLVQPGGSLRLSCAASGFTFDDYAMHWVRQAPGKGLEWVSGISWNSGSIGYADSVKGRFTISRDNAKNSLYLQMNSLRAEDTALYYCAKDSRGYGDYSRGGAYCGQGTLVTVSS

SEQ ID NO: 45 - protein, 123 aa, synthetic construct
EVQLVESGGGLVQPGRSLRLSCAASGFTFDNYAMHWVRQAPGKGLEWVSGISWNSGSIGYADSVKGRFTISRDNAKKSLYLQMNSLRAEDTALYYCAKDSRGYGDYSLGGAYWGQGTLVTVSS

SEQ ID NO: 46 - protein, 123 aa, synthetic construct
EVQLVESGGGLVQPGRSLRLSCAASGFTFDDYAMHWVRQAPGKGLEWVSGISWNSGSIGYADSVKGRFTISRDNAKKSLYLQMNSLRAEDTALYYCAKDSRGYGDYRLGGAYWGQGTLVTVSS

SEQ ID NO: 47 - protein, 123 aa, synthetic construct
EVQLVESGGGLVQPGRSLRLSCEASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 48 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGDYRLGGAYWGQGTLVTVSS

SEQ ID NO: 49 - protein, 6 aa, synthetic construct
QSVSSN

SEQ ID NO: 50 - skipped

SEQ ID NO: 51 - protein, 9 aa, synthetic construct
QQYNNWPWT

SEQ ID NO: 52 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 53 - protein, 9 aa, synthetic construct
  VARIANT 6: note = Xaa is N or S
GGSISXSYY

SEQ ID NO: 54 - protein, 14 aa, synthetic construct
  VARIANT 1: note = Xaa is T, V, or A
  VARIANT 2: note = Xaa is S or T
  VARIANT 4: note = Xaa is D or N
XXQXGVGATTTEEY

SEQ ID NO: 55 - protein, 8 aa, synthetic construct
  VARIANT 5: note = Xaa is D, A, or H
  VARIANT 6: note = Xaa is D or N
GFTFXXYA

SEQ ID NO: 56 - protein, 16 aa, synthetic construct
  VARIANT 9: note = Xaa is D or S
  VARIANT 11: note = Xaa is R or S
  VARIANT 12: note = Xaa is L or R
AKDSRGYGXYXXGGAY

SEQ ID NO: 57 - protein, 330 aa, Homo sapiens [sequence omitted]

SEQ ID NO: 58 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 13 aa, Homo sapiens
ESKYGPPCPSCPA

SEQ ID NO: 62 - protein, 13 aa, synthetic construct
ESKYGPPCPPCPA

SEQ ID NO: 63 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 64 - protein, 110 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 65 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 68 - protein, 7 aa, synthetic construct
  VARIANT 3: note = Xaa is H or Y
  VARIANT 6: note = Xaa is N or S
  VARIANT 7: note = Xaa is T or I
IYXSGXX

SEQ ID NO: 69 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 70 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

(19) United States
(12) Patent Application Publication
(10) Pub. No.: US 2023/0192843 A1
(43) Pub. Date: Jun. 22, 2023

(54) MESOTHELIN BINDING PROTEINS AND USES THEREOF

(71) Applicants: TENEOBIO, INC., Newark, CA (US); ARSENAL BIOSCIENCES, INC., South San Francisco, CA (US)

(72) Inventors: Katherine HARRIS, Fremont, CA (US); Harbani Kaur MALIK CHAUDHRY, Oakland, CA (US); Nicole ALLEN, Fremont, CA (US)

(21) Appl. No.: 18/046,213

(22) Filed: Oct. 13, 2022

Related U.S. Application Data
(60) Provisional application No. 63/255,887, filed on Oct. 14, 2021.
(60) Provisional application No. 63/255,891, filed on Oct. 14, 2021.
(60) Provisional application No. 63/303,422, filed on Jan. 26, 2022.
(60) Provisional application No. 63/392,569, filed on Jul. 27, 2022.

(51) Int. Cl. C07K 16/28; A61P 35/00

(52) U.S. Cl. CPC C07K 16/28; A61P 35/00; C07K 16/2809; A61K 39/00; C07K 2317/31; C07K 2317/622

(57) ABSTRACT

Single domain antibodies which specifically bind to mesothelin (MSLN) and mesothelin binding proteins, anti-mesothelin antibodies and antibody fragments thereof, antibody-drug conjugates, synthetic immune receptors, and diagnostic agents comprising the same are disclosed. Also disclosed are pharmaceutical compositions comprising any of the foregoing and uses of any of the foregoing in the treatment and/or diagnosis and/or monitoring of a disease associated with MSLN expression.

This application claims the benefit of priority to U.S. Provisional Application No. 63/255,887, filed Oct. 14, 2021, U.S. Provisional Application No. 63/255,891, filed Oct. 14, 2021, U.S. Provisional Application No. 63/303,422, filed Jan. 26, 2022, and U.S. Provisional Application No. 63/392,569, filed Jul. 27, 2022, the contents of each of which are incorporated by reference herein in their entireties.

Incorporated by reference in its entirety is a computer-readable amino acid sequence listing submitted concurrently herewith and identified as follows: 70,499 byte XML file named “10184-US06-SEC_Sequence_Listing”; created on Oct. 12, 2022.

Disclosed herein are single domain antibodies which specifically bind to mesothelin (MSLN) and mesothelin binding proteins, anti-mesothelin antibodies and antibody fragments thereof, antibody-drug conjugates, synthetic immune receptors, and diagnostic agents comprising the same. Pharmaceutical compositions comprising any of the foregoing and uses of any of the foregoing in the treatment and/or diagnosis and/or monitoring of a disease associated with MSLN expression are also disclosed.

Mesothelin (MSLN; also known as CAK1; UniProt Q13421; HGNC ID 7371) is a tumor-associated antigen that is broadly expressed as a cell surface glycoprotein on various malignant tumor cells. Mesothelin (MSLN) was originally identified by Pastan and colleagues in 1992 using the monoclonal antibody (mAb) K1, which was generated by the immunization of mice with human ovarian carcinoma (OVCAR-3) cells (Chang, et al., Int J. Cancer. (1992) 50:373-81). MSLN was purified from the human pancreatic cancer cell line HPC-Y5 and was shown to have megakaryocyte potentiating ability. The MSLN gene encodes a 71 kDa precursor protein that is cleaved by furin into two products: an amino-terminal shed fragment called Megakaryocyte Potentiating Factor (MPF; 31 kDa) and the glycosyl-phosphatidylinositol (GPI)-anchored glycoprotein MSLN (40 kDa), which remains attached to the cell membrane through the GPI linkage. The MSLN protein is organized into superhelical domains, with ARM-type repeats.

Although both MPF and MSLN exhibit bioactivity, their exact biological functions remain unclear. Knockout mice have been prepared in which the mesothelin gene was disrupted by homologous recombination (Bera, T. K. and Pastan, I. (2000) Mol. Cell. Biol. 20:2902-2906). No anatomical, hematologic, or reproductive abnormalities were detected, indicating that mesothelin function is not essential for growth or reproduction, at least in these knockout mice. MSLN specifically interacts with MUC16 (CA125), a mucin-like glycoprotein present on the surface of tumor cells that was previously identified as an ovarian cancer antigen. MSLN is associated with tumor cell proliferation and migration (Rump A et al., J Biol Chem. 2004 Mar 5;279(10):9190-8), and the mesothelin-MUC16 interaction has been proposed to function in cell adhesion, invasion, and metastasis. Illustratively, expression of mesothelin in the lining of the peritoneum correlates with the preferred site of metastasis formation of ovarian cancer, and mesothelin-MUC16 binding is thought to facilitate peritoneal metastasis of ovarian tumors (Gubbels, J. A. et al. (2006) Mol. Cancer. 5:50).

MSLN is highly expressed in several tumor types, including mesothelioma, pancreatic cancer, gastric cancer, ovarian cancer, lung cancer, and triple negative breast cancer (Hassan et al., Eur J Cancer (2008) 44:46-53; Ordonez, Am JSurgPathol (2003) 27:1418-28; Ho et al., Clin Cancer Res (2007) 13:1571-5). However, normal tissue expression of MSLN is limited to mesothelial cells in the pleura, pericardium, and peritoneum, as well as the surface epithelia of normal ovaries, Fallopian tubes, and tonsils, making MSLN a promising target for antibody-drug conjugates, monoclonal antibodies, and CAR-T cell therapies (Hassan R et al., J Clin Oncol. 2016 Dec;34(34):4171-4179). Moreover, MSLN binding agents can be used as a marker for the diagnosis and prognosis of certain types of cancer because trace amounts of mesothelin can be detected in the blood of some patients with mesothelin-positive cancers (Cristaudo et al., Clin. Cancer Res. 13:5076-5081, 2007). Overexpression of MSLN is associated with poor prognosis, for example, in lung adenocarcinoma and triple-negative breast cancer.

In addition to its expression on the cell surface, mesothelin is also shed into serum through the action of ADAM17/TACE. Serum levels of shed mesothelin are elevated in patients with ovarian cancer and other cancers. MESOMARK®, an ELISA test for shed serum mesothelin, is approved by the FDA for humanitarian use and may help in the diagnosis or monitoring of mesothelioma. Shed mesothelin has also been used, alone or together with other markers, to aid in diagnosis or prognosis of other cancer types. The correlation of serum levels of shed mesothelin with disease suggested a potential role for the mesothelin protein in cancer progression.

Accordingly, there is a need for additional and effective therapeutic treatments and diagnostic agents for diseases associated with mesothelin expression.

Disclosed herein is a single domain antibody which specifically binds to mesothelin (MSLN), such as, e.g., human MSLN.

In some embodiments, the single domain antibody comprises a heavy chain variable (VH) region comprising:
(i) a VH complementarity determining region one (CDR1) comprising the sequence G G S I S X₁ S Y Y (SEQ ID NO: 53), wherein X₁ is N or S;
(ii) a VH CDR2 comprising the sequence I Y X₂ S G X₃ X₄ (SEQ ID NO: 68), wherein X₂ is H or Y; X₃ is N or S; and X₄ is T or I; and
(iii) a VH CDR3 comprising the sequence X₅ X₆ Q X₇ G VG A T T TEE Y (SEQ ID NO: 54), wherein X₅ is T, V, or A; X₆ is S or T; and X₇ is D or N.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 85% (such as, e.g., 85%, 90%, 95%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 90% (such as, e.g., 90%, 95%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 95% sequence identity to the CDRs 1, 2, and 3 of any one of SEQ ID NOs: 11-19.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1 or SEQ ID NO: 9;
(ii) a VH CDR2 comprising a sequence having at most two (e.g., one, two, zero) amino acid modifications relative to SEQ ID NO: 2, SEQ ID NO: 7, or SEQ ID NO: 10; and
(iii) a VH CDR3 comprising a sequence having at most two (e.g., one, two, zero) amino acid modifications relative to SEQ ID NO: 3, SEQ ID NO: 4, SEQ ID NO: 5, SEQ ID NO: 6, or SEQ ID NO: 8.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1 or SEQ ID NO: 9. In some embodiments, the VH CDR2 comprises a sequence having at most one amino acid modification relative to SEQ ID NO: 2, SEQ ID NO: 7, or SEQ ID NO: 10. In some embodiments, the VH CDR3 comprises a sequence having at most one amino acid modification relative to SEQ ID NO: 3, SEQ ID NO: 4, SEQ ID NO: 5, SEQ ID NO: 6, or SEQ ID NO: 8. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1 and SEQ ID NO: 9. In some embodiments, the VH CDR2 comprises a sequence chosen from SEQ ID NO: 2, SEQ ID NO: 7, and SEQ ID NO: 10. In some embodiments, the VH CDR3 comprises a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(i) a VH complementarity determining region one (CDR1) comprising a sequence chosen from SEQ ID NO: 1 and SEQ ID NO: 9;
(ii) a VH CDR2 comprising a sequence chosen from SEQ ID NO: 2, SEQ ID NO: 7, and SEQ ID NO: 10; and
(iii) a VH CDR3 comprising a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(a) a VH CDR1, a VH CDR2, and a VH CDR3 comprising the sequences of SEQ ID NOs: 1, 2, and 3, respectively;
(b) a VH CDR1, a VH CDR2, and a VH CDR3 comprising the sequences of SEQ ID NOs: 1, 2, and 4, respectively;
(c) a VH CDR1, a VH CDR2, and a VH CDR3 comprising the sequences of SEQ ID NOs: 1, 2, and 5, respectively;
(d) a VH CDR1, a VH CDR2, and a VH CDR3 comprising the sequences of SEQ ID NOs: 1, 2, and 6, respectively;
(e) a VH CDR1, a VH CDR2, and a VH CDR3 comprising the sequences of SEQ ID NOs: 1, 7, and 8, respectively;
(f) a VH CDR1, a VH CDR2, and a VH CDR3 comprising the sequences of SEQ ID NOs: 9, 2, and 6, respectively;
(g) a VH CDR1, a VH CDR2, and a VH CDR3 comprising the sequences of SEQ ID NOs: 9, 2, and 4, respectively; or
(h) a VH CDR1, a VH CDR2, and a VH CDR3 comprising the sequences of SEQ ID NOs: 1, 10, and 4, respectively.

In some embodiments, the single domain antibody comprises a heavy chain variable (VH) region comprising the CDR1, CDR2, and CDR3 of any one of SEQ ID NOs: 11-19.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, at least 85%, at least 90%, at least 95%) sequence identity to any one of SEQ ID NOs: 11-19. In some embodiments, the single domain antibody comprises a heavy chain variable (VH) region having at least 85% (such as, e.g., 85%, 90%, 95%, at least 90%, at least 95%) sequence identity to any one of SEQ ID NOs: 11-19. In some embodiments, the single domain antibody comprises a heavy chain variable (VH) region having at least 90% (such as, e.g., 90%, 95%, at least 95%) sequence identity to any one of SEQ ID NOs: 11-19. In some embodiments, the single domain antibody comprises a heavy chain variable (VH) region having at least 95% sequence identity to any one of SEQ ID NOs: 11-19.

In some embodiments, the single domain antibody comprises a heavy chain variable (VH) region chosen from SEQ ID NOs: 11-19.

In some embodiments, the single domain antibody specifically binds to human MSLN. In some embodiments, the single domain antibody binds to human MSLN with a K_{D} of from about 10⁻⁹ M to about 10⁻⁶ M.

In some embodiments, the single domain antibody is an isolated single domain antibody.

Also disclosed herein is a mesothelin binding protein comprising a single domain antibody that specifically binds to mesothelin, as described herein.

In some embodiments, the mesothelin binding protein specifically binds to human MSLN. In some embodiments, the mesothelin binding protein binds to human MSLN with a K_{D} of from about 10⁻⁹ M to about 10⁻⁶ M.

In some embodiments, the mesothelin binding protein further binds to one or more target antigens other than mesothelin. In some embodiments, the mesothelin binding protein is multispecific. In some embodiments, the mesothelin binding protein is bispecific.

In some embodiments, the mesothelin binding protein further specifically binds to CD3. In some embodiments, the mesothelin binding protein further specifically binds to human CD3. In some embodiments, the mesothelin binding protein further specifically binds to human CD3 epsilon. In some embodiments, the mesothelin binding protein binds to an epitope on CD3 comprising at least one residue selected from CD3 epsilon (SEQ ID NO: 69): K73 and S83; and CD3 delta (SEQ ID NO: 70) K82 and C93. In some embodiments, the epitope on CD3 comprises the region of CD3 delta defined by K82, E83, S84, T85, V86, Q87, V88, H89, Y90, R91, M92, C93. In some embodiments, the epitope on CD3 comprises the region of CD3 epsilon defined by K73, N74, 175, G76, S77, D78, E79, D80, H81, L82, S83. In some embodiments, the epitope comprises a conformational epitope with residues of both CD3 delta and CD3 epsilon. In some embodiments, the conformational epitope comprises each of residues CD3ε K73 and S83; CD3δ K82 and C93.

In some embodiments, the mesothelin binding protein further comprises a CD3-binding VH region. In some embodiments, the mesothelin binding protein further comprises a CD3-binding VH region that is paired with a light chain (LV) region.

In some embodiments, the CD3-binding VH region comprises:
(i) a VH complementarity determining region one (CDR1) comprising a sequence having at most two (such as, e.g., zero, one, or two) amino acid modifications relative to any one of SEQ ID NOs: 20-25;
(ii) a VH CDR2 comprising a sequence having at most two (such as, e.g., zero, one, or two) amino acid modifications relative to SEQ ID NO: 26; and
(iii) a VH CDR3 comprising a sequence having at most two (such as, e.g., zero, one, or two) amino acid modifications relative to any one of SEQ ID NOs: 27-30.

In some embodiments, each amino acid modification, if any, is a conservative amino acid substitution. In some embodiments, each amino acid modification, if any, is a conservative amino acid substitution listed in Table A1.

In some embodiments, the CD3-binding VH CDR1 comprises a sequence having at most one amino acid modification relative to any one of SEQ ID NO: 20-25. In some embodiments, the CD3-binding VH CDR2 comprises a sequence having at most one amino acid modification relative to SEQ ID NO: 26. In some embodiments, the CD3-binding VH CDR3 comprises a sequence having at most one amino acid modification relative to any one of SEQ ID NOs: 27-30. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the CD3-binding VH CDR1 comprises a sequence chosen from SEQ ID NOs: 20-25. In some embodiments, the CD3-binding VH CDR2 comprises the sequence of SEQ ID NO: 26. In some embodiments, the CD3-binding VH CDR3 comprises a sequence chosen from SEQ ID NOs: 27-30.

In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 85% (such as, e.g., 85%, 90%, 95%, at least 90%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 90% (such as, e.g., 90%, 95%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 95% sequence identity to the CDRs 1, 2, and 3 of any one of SEQ ID NOs: 31-48.

In some embodiments, the CD3-binding VH region comprises the CDR1, CDR2, and CDR3 of any one of SEQ ID NOs: 31-48.

In some embodiments, the CD3-binding VH region comprises:
(i) a VH complementarity determining region one (CDR1) comprising the sequence GF TF X₈ X₉ Y A (SEQ ID NO: 55), wherein X₈ is D, A, or H and X₉ is D or N;
(ii) a VH CDR2 comprising the sequence ISWNSGSI (SEQ ID NO: 26); and
(iii) a VH CDR3 comprising the sequence A K D S R G Y G X₁₀ Y X₁₁ X₁₂ G G A Y (SEQ ID NO: 56), wherein X₁₀ is D or S; X₁₁ is R or S; and X₁₂ is L or R.

In some embodiments, the VH CDR1, VH CDR2, and VH CDR3 sequences in the CD3-binding VH region are present in a human VH framework.

In some embodiments, the CD3-binding VH region has at least 80% (such as, e.g., 80%, 85%, 90%, 95%, at least 85%, at least 90%, at least 95%) sequence identity to any one of SEQ ID NOs: 31-48. In some embodiments, the CD3-binding VH region has at least 85% (such as, e.g., 85%, 90%, 95%, at least 90%, at least 95%) sequence identity to any one of SEQ ID NOs: 31-48. In some embodiments, the CD3-binding VH region has at least 90% (such as, e.g., 90%, 95%, at least 95%) sequence identity to any one of SEQ ID NOs: 31-48. In some embodiments, the CD3-binding VH region has at least 95% sequence identity to any one of SEQ ID NOs: 31-48.

In some embodiments, the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

In some embodiments, the light chain variable region comprises the CDR1, CDR2, and CDR3 of SEQ ID NO: 52. In some embodiments, the light chain variable region comprises a VL CDR1, a VL CDR2, and a VL CDR3 comprising the sequence of SEQ ID NOs: 49, 50, and 51, respectively. In some embodiments, the VL CDR1, VL CDR2, and VL CDR3 sequences are present in a human VH framework.

In some embodiments, the light chain variable region has at least 80% (such as, e.g., 80%, 85%, 90%, 95%, at least 85%, at least 90%, at least 95%) sequence identity to SEQ ID NO: 52. In some embodiments, the light chain variable region has at least 85% (such as, e.g., 85%, 90%, 95%, at least 90%, at least 95%) sequence identity to SEQ ID NO: 52. In some embodiments, the light chain variable region has at least 90% (such as, e.g., 90%, 95%, at least 95%) sequence identity to SEQ ID NO: 52. In some embodiments, the light chain variable region has at least 95% sequence identity to SEQ ID NO: 52.

In some embodiments, the mesothelin binding protein is an anti-mesothelin antibody or fragment thereof. In some embodiments, the anti-mesothelin antibody is a monoclonal antibody or fragment thereof. In some embodiments, the anti-mesothelin antibody is an isolated monoclonal antibody or fragment thereof.

In some embodiments, the anti-mesothelin antibody is an IgG1 antibody. In some embodiments, the anti-mesothelin antibody is an IgG2 antibody. In some embodiments, the anti-mesothelin antibody is an IgG4 antibody.

In some embodiments, the mesothelin binding protein is an antibody fragment. In some embodiments, the mesothelin binding protein is a heavy chain-only antibody. In some embodiments, the mesothelin binding protein is a three-chain antibody like molecule (TCA).

In some embodiments, the anti-mesothelin antibody or fragment thereof further comprises a Fc region. In some embodiments, the anti-mesothelin antibody or fragment thereof further comprises a variant Fc region. In some embodiments, the variant Fc region comprises heterodimerizing alterations. In some embodiments, the Fc region is a silenced Fc region.

Also disclosed herein is a polynucleotide encoding a single domain antibody that specifically binds to mesothelin as described herein.

Also disclosed herein is a composition comprising one or more polynucleotide(s) encoding a mesothelin binding protein as described herein. In some embodiments, the mesothelin binding protein is an anti-mesothelin antibody or fragment thereof.

Also disclosed herein is a recombinant expression vector comprising a single domain antibody that specifically binds to mesothelin as described herein, as well as a host cell comprising the recombinant expression vector.

Also disclosed herein is one or more recombinant expression vector(s) comprising one or more polynucleotide(s) encoding a mesothelin binding protein as described herein, as well as a host cell comprising the one or more recombinant expression vector(s).

Also disclosed herein is a synthetic immune receptor comprising a single domain antibody that specifically binds to mesothelin as described herein, as well as a cell comprising the synthetic immune receptor.

Also disclosed herein is an antibody-drug conjugate comprising a single domain antibody that specifically binds to mesothelin, as described herein. In some embodiments, the antibody-drug conjugate is for use in a diagnostic application, such as, e.g., the detection or monitoring of a disease associated with mesothelin expression, such as, e.g., a proliferative disease or cancer.

Also disclosed herein is a pharmaceutical composition comprising a mesothelin binding protein, antibody-drug conjugate, or anti-mesothelin antibody or fragment thereof and a pharmaceutically acceptable excipient.

Also disclosed herein is a method of treating a disease associated with mesothelin expression in a subject in need thereof comprising administering to the subject a therapeutically effective dose of at least one mesothelin binding protein, antibody-drug conjugate, anti-mesothelin antibody, or antibody fragment as described herein. In some embodiments, the disease associated with mesothelin expression is chosen from proliferative diseases and cancer. In some embodiments, the cancer is chosen from mesothelioma, pancreatic cancer, gastric cancer, ovarian cancer, lung cancer, and triple negative breast cancer.

Also disclosed herein is a mesothelin binding protein, antibody-drug conjugate, anti-mesothelin antibody, or antibody fragment as described herein for use in the treatment of a disease associated with mesothelin expression. In some embodiments, the disease associated with mesothelin expression is chosen from proliferative diseases and cancer. In some embodiments, the cancer is chosen from mesothelioma, pancreatic cancer, gastric cancer, ovarian cancer, lung cancer, and triple negative breast cancer.

Also disclosed herein is a use of a mesothelin binding protein, antibody-drug conjugate, anti-mesothelin antibody, or antibody fragment as described herein in the manufacture of a medicament for the treatment of a disease associated with mesothelin expression. In some embodiments, the disease associated with mesothelin expression is chosen from proliferative diseases and cancer. In some embodiments, the cancer is chosen from mesothelioma, pancreatic cancer, gastric cancer, ovarian cancer, lung cancer, and triple negative breast cancer.

### BRIEF DESCRIPTION OF THE DRAWINGS

FIG. 1A depicts representative CHO cell binding dose curves for example single domain antibodies of the present disclosure, where the CHO cells express human MSLN. FIG. 1B depicts representative HeLa cell binding dose curves for example single domain antibodies of the present disclosure. FIG. 1C depicts representative CHO cell binding dose curves for example single domain antibodies of the present disclosure, where the CHO cells do not express MSLN protein. In FIGS. 1A-1C, PE mean fluorescence intensity was plotted as a fold over background (i.e., cells incubated with secondary detection antibody only).

FIG. 2A is a schematic illustration of a CAR-T structure comprising an anti-MSLN extracellular binding domain comprising an antibody sequence described herein.

FIG. 2B depicts T-cell activity of Jurkat cells transfected an anti-MSLN 394556 CAR with CHO-huMSLN (**p= 0.0075) and HeLa (**p=0.0015).

### DEFINITIONS

In some embodiments, “about,” when used in connection with a measurable numerical variable, refers to the indicated value of the variable and to all values of the variable that are within the experimental error of the indicated value (e.g., within the 95% confidence interval for the mean) or ± 10% of the indicated value, whichever is greater. In some embodiments, numeric ranges are inclusive of the numbers defining the range (i.e., the endpoints).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “antibody” generally refers to a tetrameric immunoglobulin protein comprising two light chain polypeptides (such as, e.g., light chain polypeptides that are about 25 kDa each) and two heavy chain polypeptides (such as, e.g., heavy chain polypeptides that are about 50-70 kDa each). The term “light chain” or “immunoglobulin light chain,” as used herein, refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (µ), delta (Δ), gamma (γ), alpha (α), and epsilon (ε), and define the antibody’s isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains. In some embodiments, antibodies of the present disclosure are human antibodies or humanized antibodies and can be of the IgG1-, IgG2-, IgG3-, or IgG4-type.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., MSLN or CD3). From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001). In some embodiments of the present disclosure, “CDR” means a complementarity-determining region of an antibody as defined in Lefranc, MP et al., IMGT, the International ImMunoGeneTics database, Nucleic Acids Res., 27:209-212 (1999).

“Framework Region” or “FR” residues are those variable domain residues other than the hypervariable region/CDR residues as herein defined.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies, single domain antibodies, antibody fragments, and the like mean residue numbering by the EU numbering system.

As used herein, an “anti-mesothelin antibody” is an antibody that specifically binds to mesothelin (MSLN).

The term “monoclonal antibody,” as used herein, refers to an antibody obtained from a population of substantially homogeneous antibodies, i.e., the individual antibodies comprising the population are identical except for possible naturally occurring mutations that may be present in minor amounts. In contrast to polyclonal antibody preparations which typically include different antibodies directed against different determinants (epitopes), a monoclonal antibody is generally directed against a single determinant on the antigen. As non-limiting examples, monoclonal antibodies in accordance with the present disclosure can be made by the hybridoma method first described by Kohler et al. (1975) Nature 256:495, and can also be made via recombinant protein production methods (see, e.g., U.S. Pat. No. 4,816,567).

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences that are derived from the germline of another mammalian species, such as, e.g., a mouse, have been grafted onto human framework sequences.

As used herein, an “antibody fragment” generally refers to a fragment of a full-length antibody, such as, e.g., VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain) or a modified fragment of a full-length antibody, such as, e.g., triple-chain antibody-like molecule, heavy-chain only antibody, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂ , ((scFv)₂-CH3 + CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

As used herein, the term “heavy chain-only antibody” refers to a dimeric immunoglobulin protein consisting of two heavy chain polypeptides (such as, e.g., heavy chain polypeptides that are about 50-70 kDa each). A “heavy chain-only antibody” is an antibody fragment that lacks the two light chain polypeptides found in a conventional antibody. In some embodiments, a “heavy chain-only antibody” is a homodimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, a “single domain antibody” refers to a single polypeptide chain that contains all or part of the heavy chain variable domain or all or part of the light chain variable domain of an antibody. In some embodiments, the single domain antibody is a human single domain antibody.

As used herein, the term “three-chain antibody like molecule” or “TCA” refers to antibody-like molecules comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

As used herein, an “antigen-binding fragment” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid.

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. In some embodiments of the present disclosure, a mesothelin binding protein (such as, e.g., an anti-mesothelin antibody fragment, such as, e.g., a TCA or heavy chain-only antibody) comprises an Fc region from an immunoglobulin. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “functional Fc region” possesses an “effector function” of a native-sequence Fc region. Non-limiting examples of effector functions include C1q binding, CDC; Fc-receptor binding, ADCC, ADCP, down-regulation of cell-surface receptors (e.g., B-cell receptor), etc. Such effector functions generally require the Fc region to interact with a receptor, such as, e.g., the FcyRI; FcyRIIA; FcγRIIB1; FcyRIIB2; FcyRIIIA; FcyRIIIB receptors, and the low affinity FcRn receptor; and can be assessed using various assays known in the art.

A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, for example, one or more (e.g., two or more, three or more, four or more) amino acid substitution(s). Illustratively, in some embodiments, the variant Fc region has at least one amino acid substitution compared to a native-sequence Fc region or to the Fc region of a parent polypeptide, e.g., from about one to about ten amino acid substitutions, e.g., from about one to about five amino acid substitutions in a native-sequence Fc region or in the Fc region of the parent polypeptide. In some embodiments, the variant Fc region herein will possess at least about 80% homology with a native-sequence Fc region and/or with an Fc region of a parent polypeptide, e.g., at least about 85% homology therewith, e.g., at least about 90% homology therewith, e.g., at least about 95% homology therewith, e.g., at least about 99% homology therewith.

As used herein, “heterodimerizing alterations” refer to alterations in the A and B chains of an Fc region (i.e., the two chains comprising the Fc region, wherein one chain is referred to herein as the “A” chain and the other is referred to herein as the “B” chain) that facilitate the formation of heterodimeric Fc regions, that is, Fc regions in which the A chain and the B chain of the Fc region do not have identical amino acid sequences. In some embodiments, heterodimerizing alterations can be asymmetric, that is, an A chain having a certain alteration can pair with a B chain having a different alteration. These alterations facilitate heterodimerization and disfavor homodimerization. Whether hetero- or homo-dimers have formed can be assessed, for example, by size differences as determined by polyacrylamide gel electrophoresis in situations where one polypeptide chain is a dummy Fc and the other is an scFv-Fc. One non-limiting example of such paired heterodimerizing alterations are the so-called “knobs and holes” substitutions. See, e.g., U.S. Pat. No. 7,695,936 and U.S. Pat. Application Publication No. 2003/0078385. As used herein, an Fc region that comprises one pair of knobs and holes substitutions, comprises one substitution in the A chain and another in the B chain. For example, the following knobs and holes substitutions in the A and B chains of an IgG1 Fc region have been found to increase heterodimer formation as compared with that found with unmodified A and B chains and may be employed in non-limiting embodiments of this disclosure: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. Alternatively or in addition to such alterations, substitutions creating new disulfide bridges can facilitate heterodimer formation. See, e.g., U.S. Patent Application Publication No. 2003/0078385. Such alterations in an IgG1 Fc region include, but are not limited to, the following substitutions: Y349C in one Fc polypeptide chain and S354C in the other; Y349C in one Fc polypeptide chain and E356C in the other; Y349C in one Fc polypeptide chain and E357C in the other; L351C in one Fc polypeptide chain and S354C in the other; T394C in one Fc polypeptide chain and E397C in the other; or D399C in one Fc polypeptide chain and K392C in the other. Additionally or alternatively, substitutions changing the charge of a one or more residue(s), for example, in the CH3-CH3 interface, can enhance heterodimer formation, as described, for example, in WO 2009/089004, which is incorporated by reference herein. Such substitutions are referred to herein as “charge pair substitutions,” and an Fc region comprising one pair of charge pair substitutions comprises one substitution in the A chain and a different substitution in the B chain. Non-limiting examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of these heterodimerizing alterations can be used in polypeptides comprising variant Fc regions as described herein.

In some non-limiting embodiments, variant Fc sequences may include three amino acid substitutions in the CH2 region to reduce FcyRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour KL. et al., 1999 Eur J Immunol. 29(8):2613-24; and Shields R.L. et al., 2001. JBiol Chem. 276(9):6591-604). The human IgG4 Fc amino acid sequence (UniProtKB No. P01861) is provided herein as SEQ ID NO: 76. Silenced IgG1 is described, for example, in Boesch, A.W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting, or adding amino acid residues to effect complement binding or Fc receptor binding. For example, and not by way of limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Antibodies and antibody fragments with reduced effector function include, but are not limited to, those with substitution of one or more of Fc region residues 238, 265, 269, 270, 297, 327 and 329 according to EU numbering (see, e.g., U.S. Pat. No. 6,737,056). In some embodiments, variant Fc regions with reduced effector function comprise substitutions at two or more of amino acid positions 265, 269, 270, 297 and 327 according to EU numbering, including the so-called “DANA” Fc mutant with substitution of residues 265 and 297 to alanine according to EU numbering (i.e., D265A and N297A according to EU numbering) (see, e.g., U.S. Pat. No. 7,332,581). In some embodiments, the variant Fc region with reduced effector function comprises the following two amino acid substitutions: D265A and N297A.

In some embodiments, effector function is reduced through a mutation in a constant region that eliminates glycosylation, e.g., an “effector-less mutation.” In some embodiments, the effector-less mutation is an N297A or a DANA mutation (D265A+N297A) in the CH2 region. Shields et al., J. Biol. Chem. 276 (9): 6591-6604 (2001). In some embodiments, the effector-less mutation is an N297G or a DANG mutation (D265A+N297G) in the CH2 region. In some embodiments, the variant Fc region lacks glycosylation at N297, e.g., the variant Fc region is a variant Fc region lacking glycosylation at N297 as described in International Patent Publication No. WO 2014/153063, which is incorporated by reference herein. Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in host cells which result in an altered glycolsylation pattern that is ineffective or less effective at promoting effector function (e.g., Shinkawa et al., J. Biol. Chem. 278(5): 3466-3473 (2003)).

In some embodiments, the proline at position 329 (EU numbering) (P329) of a wild-type human Fc region is substituted with glycine or arginine or an amino acid residue large enough to destroy the proline sandwich within the Fc/Fcy receptor interface, that is formed between the P329 of the Fc and tryptophan residues W87 and W110 of FcgRIII (Sondermann et al., Nature 406, 267-273 (20 Jul. 2000)). In some further embodiments, at least one further amino acid substitution in the Fc variant region is S228P, E233P, L234A, L235A, L235E, N297A, N297D, or P331S. In some embodiments, the at least one further amino acid substitution is L234A and L235A of the human IgG1 Fc region or S228P and L235E of the human IgG4 Fc region, all according to EU numbering (see, e.g., U.S. Pat. No. 8,969,526, which is incorporated by reference in its entirety).

In some embodiments, the variant Fc region has P329 of the human IgG Fc region substituted with glycine, wherein the variant Fc region comprises at least two further amino acid substitutions at L234A and L235A of the human IgG1 Fc region or S228P and L235E of the human IgG4 Fc region, and wherein the residues are numbered according to the EU numbering (see, e.g., U.S. Pat. No. 8,969,526). In some embodiments, the variant Fc region comprising the P329G, L234A and L235A (EU numbering) substitutions exhibits a reduced affinity to the human FcyRIIIA and FcyRIIA.

In some embodiments, the variant Fc region comprises a triple mutation: an amino acid substitution at position P329, a L234A, and a L235A mutation according to EU numbering (P329/LALA) (see, e.g., U.S. Pat. No. 8,969,526). In some embodiments, the variant Fc region comprises the following amino acid substitutions: P329G, L234A, and L235A according to EU numbering.

In some embodiments, an antibody or antibody fragment comprises a variant human IgG4 CH3 domain sequence comprising a T366W mutation, which can optionally be referred to herein as an IgG4 CH3 knob sequence. In some embodiments, an antibody or antibody fragment comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody or antibody fragment comprises a heavy chain polypeptide subunit comprising a variant human IgG4 Fc region comprising an S228P mutation, an F234A mutation, an L235A mutation, and a T366W mutation (knob). In some embodiments, an antibody or antibody fragment comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Pat. Publication Nos. 2005/0136049 and 2005/0037421. Alternative scaffolds can be made from human variable-like domains that more closely match the shark V-NAR scaffold and may provide a framework for a long penetrating loop structure.

As used herein, the term “antigen binding protein” refers to a protein that specifically binds to one or more target antigens. An antigen binding protein typically comprises an antigen-binding fragment that specifically binds to an antigen and, optionally, a scaffold or framework portion that allows the antigen-binding fragment to adopt a conformation that promotes binding of the antigen binding protein to the antigen. In some embodiments, an antigen binding protein is an antibody or antibody fragment. In some embodiments, an antigen binding protein is a protein comprising one or more antigen-binding fragments incorporated into a single polypeptide chain or into multiple polypeptide chains. For instance, antigen binding proteins can include, but are not limited to, a diabody (see, e.g., EP 404,097; WO 93/11161; and HoUinger etal, Proc. Natl. Acad. Sci. USA, Vol. 90:6444-6448, 1993); an intrabody; a domain antibody (single VL or VH domain or two or more VH domains joined by a peptide linker; see Ward et al, Nature, Vol. 341:544-546, 1989); a maxibody (2 scFvs fused to Fc region, see Fredericks et al, Protein Engineering, Design & Selection, Vol. 17:95-106, 2004 and Powers etal, Journal of Immunological Methods, Vol. 251:123-135, 2001); atriabody; a tetrabody; a minibody (scFv fused to CH3 domain; see Olafsen et ah, Protein Eng Des Sel. , Vol.17:315-23, 2004); a peptibody (one or more peptides attached to an Fc region, see WO 00/24782); a linear antibody (a pair of tandem Fd segments (VH-CHl-VH-CHl) which, together with complementary light chain polypeptides, form a pair of antigen binding regions, see Zzjpate. et al, Protein Eng., Vol. 8: 1057-1062, 1995); a small modular immunopharmaceutical (see U.S. Pat. Publication No. 20030133939); and immunoglobulin fusion proteins (e.g. IgG-scFv, IgG-Fab, 2scFv-IgG, 4scFv-lgG, VH-IgG, IgG-VH, and Fab-scFv-Fc; see, e.g., Spiess et al, Mol. Immunol., Vol. 67(2 Pt A):95-106, 2015).

As used herein, a “mesothelin binding protein” is an antigen binding protein that specifically binds to mesothelin. In some embodiments, a mesothelin binding protein may also bind to one or more target antigens other than mesothelin.

Antibodies and antibody fragments (such as, e.g., heavy chain-only antibodies and three-chain antibody like molecules) of the present disclosure include multi-specific antibodies and antibody fragments, which are antibodies and antibody fragments having more than one binding specificity. As used herein, the term “multi-specific” includes “bispecific” (i.e., two binding specificities) and “trispecific” (i.e., three binding specificities), as well as higher-order independent specific binding affinities, such as higher-order polyepitopic specificity.

As used herein, an “isolated” molecule (such as, e.g., an antibody, antibody fragment, single domain antibody, mesothelin binding protein) is a molecule which has been identified and separated and/or recovered from a component of its natural environment. Contaminant components of its natural environment are materials which may interfere with diagnostic or therapeutic uses for the molecule, such as, e.g., enzymes, hormones, and other proteinaceous or nonproteinaceous solutes. In some embodiments, the isolated molecule will be purified (1) to greater than 95% by weight of the molecule as determined by the Lowry method, such as, e.g., more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, e.g., silver stain. In some embodiments, an isolated molecule will be prepared by a process comprising at least one purification step.

As used herein, an “antibody-drug conjugate” refers to an antibody or antibody fragment which is coupled to another moiety, such as, e.g., a payload, such as, e.g., a radionuclide.

As used herein, an “epitope” is a site on the surface of an antigen molecule to which a single antibody or antibody fragment binds. Generally, an antigen has several or many different epitopes and reacts with many different antibodies and antibody fragments. The term specifically includes linear epitopes and conformational epitopes. Conformational and nonconformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding, such as, e.g., amino acid residues which are effectively blocked by the specifically antigen binding peptide (in other words, the amino acid residue is within the footprint of the specifically antigen binding peptide).

As used herein, “polyepitopic specificity” refers to the ability to specifically bind to two or more different epitopes on the same or different target(s).

The terms “subject,” “individual,” and “patient” are used interchangeably herein to refer to a mammal being assessed for treatment and/or being treated. Subjects may be human, but also include other mammals, such as, e.g., those mammals useful as laboratory models for human disease, such as, e.g., mouse, rat, etc. In some embodiments, the mammal is a human.

As used herein, the term “treatment” encompasses any improvement of a disease in the subject, including the slowing or stopping of the progression of a disease in the subject, a decrease in the number or severity of the symptoms of the disease, or an increase in frequency or duration of periods where the patient is free from the symptoms of the disease.

As used herein, the term “effector cell” refers to an immune cell which is involved in the effector phase of an immune response, as opposed to the cognitive and activation phases of an immune response. Example immune cells include a cell of a myeloid or lymphoid origin, for instance, lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express specific Fc receptors (FcRs) and carry out specific immune functions. In some embodiments, an effector cell is capable of inducing ADCC, such as a natural killer cell. For example, monocytes, macrophages, which express FcRs are involved in specific killing of target cells and presenting antigens to other components of the immune system, or binding to cells that present antigens.

As used herein, the term “vector” refers to a nucleic acid molecule capable of transporting another nucleic acid to which it has been linked. One type of vector is a “plasmid,” which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (such as, e.g., bacterial vectors having a bacterial origin of replication and episomal mammalian vectors). Other vectors (such as, e.g., non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors.” In some embodiments, expression vectors for use in recombinant DNA techniques are in the form of plasmids.

As used herein, the term “host cell” refers to a cell into which an expression vector has been introduced. It should be understood that “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Example recombinant host cells include, but are not limited to, transfectomas, such as CHO cells, HEK293 cells, NS/0 cells, and lymphocytic cells.

The term “K_{D}” (M), as used herein, refers to the dissociation equilibrium constant of a particular antigen binding interaction as determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kₒₙ). Antibody dissociation rates (k_{off}) are measured in the final step, where the sensors are dipped into wells containing buffer only. The K_{D} is the ratio of k_{off}/k_{on.} (For further details see, Concepcion, J, et al., Comb Chem High Throughput Screen, 12(8), 791-800, 2009).

As used herein, a molecule (such as, e.g., a protein, antibody, or antibody fragment) “specifically binds” to a target antigen when it has a significantly higher binding affinity for, and consequently is capable of distinguishing, that antigen compared to its affinity for other unrelated proteins, under similar binding assay conditions. Molecules that specifically bind an antigen may bind to that antigen with an equilibrium dissociation constant (K_{D}) ≤ 1 × 10⁻⁶ M. Molecules specifically bind antigen with “high affinity” when the K_{D} is ≤ 1 × 10⁻⁸ M. In some embodiments, molecules described herein bind to human MSLN and/or human CD3 with a K_{D} of ≤ 5 × 10⁻⁷ M. In some embodiments, molecules described herein bind to human MSLN and/or human CD3 with a K_{D} of ≤ 1 × 10⁻⁷ M. In some embodiments, molecules described herein bind to human MSLN and/or human CD3 with a K_{D} of ≤ 5 × 10⁻⁸ M. In some embodiments, molecules described herein bind to human MSLN and/or human CD3 with a K_{D} of ≤ 2 × 10⁻⁸ M. In some embodiments, molecules described herein bind to human MSLN and/or human CD3 with a K_{D} of ≤ 1 × 10⁻⁸ M. In some embodiments, molecules described herein bind to human MSLN and/or human CD3 with a K_{D} of ≤ 1 × 10⁻⁹ M.

Affinity may be determined using a variety of techniques, a non-limiting example of which is an affinity ELISA assay. In some embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D} x kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method.

As used herein, an “[Y]-binding VH CDR” refers to a CDR of a VH region, wherein the VH region specifically binds to the target [Y].

As used herein, the term “amino acid” or “amino acid residue” refers to an amino acid having its art recognized definition, such as, e.g., an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

As used herein, “amino acid modifications” include, but are not limited to, deletions from, and/or insertions into, and/or substitutions of, residues within an amino acid sequence. Any combination of deletion, insertion, and substitution may be made to arrive at a final construct, provided that the final construct possesses the desired characteristics. The amino acid changes also may alter post-translational processes of the antibody constructs, such as changing the number or position of glycosylation sites. Preferred substitutions (or replacements) are conservative substitutions. However, any substitution (including non-conservative substitutions) is envisaged as long as the final construct retains its capability to bind to the target antigen.

One of skill in the art will realize that conservative variants of the antibodies and antibody fragments described herein can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. In some embodiments, amino acid substitutions (such as, e.g., at most one, at most two, at most three, at most four, or at most five amino acid substitutions) can be made in the V_{H} and/or the V_{L} regions to increase yield. Conservative amino acid substitution tables providing functionally similar amino acids are well known to one of ordinary skill in the art, such as, e.g., those described in Table A1.

TABLE A1

Example Conservative Substitutions

| Original | Example Substitutions | Specific Example Substitutions |
|---|---|---|
| Ala (A) | Val, Leu, Ile | Val |
| Arg (R) | Lys, Gln, Asn | Lys |
| Asn (N) | Gln, His, Asp, Lys, Arg | Gln |
| Original | Example Substitutions | Specific Example Substitutions |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, a “synthetic immune receptor” is an artificial cell receptor (such as, e.g., an artificial T cell receptor, an artificial NK cell receptor) that is engineered to be expressed on an immune effector cell and specifically bind a target antigen.

As used herein, “percent (%) amino acid sequence identity” or “percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

The term “pharmaceutical composition” refers to a preparation which is in such form as to permit the biological activity of the active ingredient to be effective, and which contains no additional components which are unacceptably toxic to a subject to which the formulation would be administered. Such compositions are sterile. “Pharmaceutically acceptable” excipients (e.g., vehicles, additives) are those which can reasonably be administered to a subject mammal to provide an effective dose of the active ingredient employed.

As used herein, a “sterile” composition is aseptic or free or essentially free from all living microorganisms and their spores. As used herein, a “frozen” composition is one at a temperature below 0° C.

As used herein, a “stable” composition is one in which the protein therein essentially retains its physical stability and/or chemical stability and/or biological activity upon storage. In some embodiments, the composition essentially retains its physical and chemical stability, as well as its biological activity upon storage. The storage period is generally selected based on the intended shelf-life of the composition. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example, using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

Some embodiments of the present disclosure relate to a single domain antibody which specifically binds to mesothelin (MSLN). Specifically, the present disclosure provides a family of closely related single domain antibodies that specifically bind to human mesothelin (MSLN). The single domain antibodies of this family comprise a set of CDR sequences as defined herein and as shown in Tables S1 and S2, and are exemplified by the provided heavy chain variable region (VH) sequences of SEQ ID NOs: 11-19 as set forth in Table S3. This family of single domain antibodies provides a number of benefits that contribute to their utility as clinically therapeutic agent(s). Illustratively, the single domain antibodies include members with a range of binding affinities, allowing the selection of a specific sequence with a desired binding affinity.

TABLE S1

Anti-MSLN Heavy Chain Antibody CDR1, CDR2 and CDR3 Amino Acid Sequences

| Clone ID # | SEQ_aa_CDR1 | SEQ_aa_CDR2 | SEQ_aa_CDR3 |
|---|---|---|---|
| 394556 | GGSISNSYY (SEQ ID NO: 1) | IYHSGNT (SEQ ID NO: 2) | VTQDGVGATTTEEY (SEQ ID NO: 3) |
| 394541 | GGSISNSYY (SEQ ID NO: 1) | IYHSGNT (SEQ ID NO: 2) | TSQDGVGATTTEEY (SEQ ID NO: 4) |
| 394582 | GGSISNSYY (SEQ ID NO: 1) | IYHSGNT (SEQ ID NO: 2) | TTQNGVGATTTEEY (SEQ ID NO: 5) |
| 392026 | GGSISNSYY (SEQ ID NO: 1) | IYHSGNT (SEQ ID NO: 2) | TSQDGVGATTTEEY (SEQ ID NO: 4) |
| 394573 | GGSISNSYY (SEQ ID NO: 1) | IYHSGNT (SEQ ID NO: 2) | ATQNGVGATTTEEY (SEQ ID NO: 6) |
| 394607 | GGSISNSYY (SEQ ID NO: 1) | IYYSGST (SEQ ID NO: 7) | ATQDGVGATTTEEY (SEQ ID NO: 8) |
| 394606 | GGSISSSYY (SEQ ID NO: 9) | IYHSGNT (SEQ ID NO: 2) | ATQNGVGATTTEEY (SEQ ID NO: 6) |
| 394610 | GGSISSSYY (SEQ ID NO: 9) | IYHSGNT (SEQ ID NO: 2) | TSQDGVGATTTEEY (SEQ ID NO: 4) |
| 394567 | GGSISNSYY (SEQ ID NO: 1) | IYYSGSI (SEQ ID NO: 10) | TSQDGVGATTTEEY (SEQ ID NO: 4) |

TABLE S2

Anti-MSLN Heavy Chain Antibody Unique CDR Amino Acid Sequences

| | | |
|---|---|---|
| SEQ_aa_CDR1 | SEQ_aa_CDR2 | SEQ_aa_CDR3 |
| GGSISNSYY (SEQ ID NO: 1) | IYHSGNT (SEQ ID NO: 2) | VTQDGVGATTTEEY (SEQ ID NO: 3) |
| GGSISSSYY (SEQ ID NO: 9) | IYYSGST (SEQ ID NO: 7) | TSQDGVGATTTEEY (SEQ ID NO: 4) |
| | IYYSGSI (SEQ ID NO: 10) | TTQNGVGATTTEEY (SEQ ID NO: 5) |
| | | ATQNGVGATTTEEY (SEQ ID NO: 6) |
| | | ATQDGVGATTTEEY (SEQ ID NO: 8) |

TABLE S3

Anti-MSLN Heavy Chain Antibody Variable Domain Amino Acid Sequences

| Clone ID # | SEQ_aa_FR1_FR4 | SEQ ID NO. |
|---|---|---|
| 394556 | QLQLQESGPGLVKPSETLSLTCTVSGGSISNSYYWGWIRQPPGKG LEWIGSIYHSGNTYYNPSLKSRVTISVDTSKNQFSLKLSSVTAADT AVYYCVTQDGVGATTTEEYWGQGTLVTVSS | 11 |
| 394541 | QLQLQESGPGLVKPSETLSLTCTVSGGSISNSYYWGWIRQPPGKG LEWIGSIYHSGNTYYNPSLKSRVTMSVDTSKNQFSLKLSSVTAAD TAVYYCTSQDGVGATTTEEYWGQGTLVTVSS | 12 |
| 394582 | QLQLQESGPGLVKPSETLSLTCTVSGGSISNSYYWGWIRQPPGKG LEWIGSIYHSGNTYYNPSLKSRVTISVDTSKNQFSLKLSSVTAADT AVYYCTTQNGVGATTTEEYWGQGTLVTVSS | 13 |
| 392026 | QLQLQESGPGLVKPSETLSLTCTVSGGSISNSYYWGWIRQPPGKG LEWIGSIYHSGNTYYNPSLKSRVTISVDTSKNQFSLKLSSVTAADT AVYYCTSQDGVGATTTEEYWGQGTLVTVSS | 14 |
| 394573 | QLQLQESGPGLVKPSETLSLTCTVSGGSISNSYYWGWIRQPPGKG LEWIGSIYHSGNTYYNPSLKSRVTISVDTSKNQFSLKLSSVTAADT AVYYCATQNGVGATTTEEYWGQGTLVTVSS | 15 |
| 394607 | QLQLQESGPGLVKPSETLSLTCTVSGGSISNSYYWGWIRQPPGKG LEWIGSIYYSGSTYYNPSLKSRVTISVDTSKNQFSLSLNSVTAADT AVYYCATQDGVGATTTEEYWGQGTLVTVSS | 16 |
| 394606 | QLQLQESGPGLVKPSETLSLTCTVSGGSISSSYYWGWIRQPPGKG LEWIGSIYHSGNTYYNPSLKSRVTISVDTSKNQFSLKLSSVTAADT AVYYCATQNGVGATTTEEYWGQGTLVTVSS | 17 |
| 394610 | QLQLQESGPGLVKPSETLSLTCTVSGGSISSSYYWGWIRQPPGKG LEWIGSIYHSGNTYYNPSLKSRVTISVDTSKNQFSLKLSSVTAADT AVYYCTSQDGVGATTTEEYWGQGTLVTVSS | 18 |
| 394567 | QLQLQESGPGLVKPSETLSLTCTVSGGSISNSYYWGWIRQPPGKG LEWIGSIYYSGSIHYNPSLKSRVTISVDTSKNQFSLKLSSVTAADT AVYYCTSQDGVGATTTEEYWGQGTLVTVSS | 19 |

In some embodiments, the single domain antibody comprises a heavy chain variable (VH) region comprising:
(i) a VH complementarity determining region one (CDR1) comprising the sequence G G S I S X₁ S Y Y (SEQ ID NO: 53), wherein X₁ is N or S;
(ii) a VH CDR2 comprising the sequence I Y X₂ S G X₃ X₄ (SEQ ID NO: 68), wherein X₂ is H or Y; X₃ is N or S; and X₄ is T or I; and
(iii) a VH CDR3 comprising the sequence X₅ X₆ Q X₇ G VG A T T TEE Y (SEQ ID NO: 54), wherein X₅ is T, V, or A; X₆ is S or T; and X₇ is D or N.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 85% (such as, e.g., 85%, 90%, 95%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 90% (such as, e.g., 90%, 95%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 95% sequence identity to the CDRs 1, 2, and 3 of any one of SEQ ID NOs: 11-19.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 11. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 12. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 13. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 14. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 15. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 16. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 17. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 18. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 19.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1 or SEQ ID NO: 9;
(ii) a VH CDR2 comprising a sequence having at most two (e.g., one, two, zero) amino acid modifications relative to SEQ ID NO: 2, SEQ ID NO: 7, or SEQ ID NO: 10; and
(iii) a VH CDR3 comprising a sequence having at most two (e.g., one, two, zero) amino acid modifications relative to SEQ ID NO: 3, SEQ ID NO: 4, SEQ ID NO: 5, SEQ ID NO: 6, or SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 3.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 4.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 5.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 6.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 7; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 9; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 6.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 9; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 4.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 10; and (iii) a VH CDR3 comprising a sequence having at most two (e.g., one, two, zero) amino acid modifications relative to SEQ ID NO: 4.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1 or SEQ ID NO: 9. In some embodiments, the VH CDR2 comprises a sequence having at most one amino acid modification relative to SEQ ID NO: 2, SEQ ID NO: 7, or SEQ ID NO: 10. In some embodiments, the VH CDR3 comprises a sequence having at most one amino acid modification relative to SEQ ID NO: 3, SEQ ID NO: 4, SEQ ID NO: 5, SEQ ID NO: 6, or SEQ ID NO: 8. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1 and SEQ ID NO: 9. In some embodiments, the VH CDR2 comprises a sequence chosen from SEQ ID NO: 2, SEQ ID NO: 7, and SEQ ID NO: 10. In some embodiments, the VH CDR3 comprises a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(i) a VH complementarity determining region one (CDR1) comprising a sequence chosen from SEQ ID NO: 1 and SEQ ID NO: 9;
(ii) a VH CDR2 comprising a sequence chosen from SEQ ID NO: 2, SEQ ID NO: 7, and SEQ ID NO: 10; and
(iii) a VH CDR3 comprising a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(a) a VH CDR1, a VH CDR2, and a VH CDR3 comprising the sequences of SEQ ID NOs: 1, 2, and 3, respectively;
(b) a VH CDR1, a VH CDR2, and a VH CDR3 comprising the sequences of SEQ ID NOs: 1, 2, and 4, respectively;
(c) a VH CDR1, a VH CDR2, and a VH CDR3 comprising the sequences of SEQ ID NOs: 1, 2, and 5, respectively;
(d) a VH CDR1, a VH CDR2, and a VH CDR3 comprising the sequences of SEQ ID NOs: 1, 2, and 6, respectively;
(e) a VH CDR1, a VH CDR2, and a VH CDR3 comprising the sequences of SEQ ID NOs: 1, 7, and 8, respectively;
(f) a VH CDR1, a VH CDR2, and a VH CDR3 comprising the sequences of SEQ ID NOs: 9, 2, and 6, respectively;
(g) a VH CDR1, a VH CDR2, and a VH CDR3 comprising the sequences of SEQ ID NOs: 9, 2, and 4, respectively; or
(h) a VH CDR1, a VH CDR2, and a VH CDR3 comprising the sequences of SEQ ID NOs: 1, 10, and 4, respectively.

In some embodiments, the single domain antibody comprises a heavy chain variable (VH) region comprising a VH CDR1, a VH CDR2, and a VH CDR3 comprising the sequences of SEQ ID NOs: 1, 2, and 3, respectively. In some embodiments, the single domain antibody comprises a heavy chain variable (VH) region comprising a VH CDR1, a VH CDR2, and a VH CDR3 comprising the sequences of SEQ ID NOs: 1, 2, and 4, respectively. In some embodiments, the single domain antibody comprises a heavy chain variable (VH) region comprising a VH CDR1, a VH CDR2, and a VH CDR3 comprising the sequences of SEQ ID NOs: 1, 2, and 5, respectively. In some embodiments, the single domain antibody comprises a heavy chain variable (VH) region comprising a VH CDR1, a VH CDR2, and a VH CDR3 comprising the sequences of SEQ ID NOs: 1, 2, and 6, respectively. In some embodiments, the single domain antibody comprises a heavy chain variable (VH) region comprising a VH CDR1, a VH CDR2, and a VH CDR3 comprising the sequences of SEQ ID NOs: 1, 7, and 8, respectively. In some embodiments, the single domain antibody comprises a heavy chain variable (VH) region comprising a VH CDR1, a VH CDR2, and a VH CDR3 comprising the sequences of SEQ ID NOs: 9, 2, and 6, respectively. In some embodiments, the single domain antibody comprises a heavy chain variable (VH) region comprising a VH CDR1, a VH CDR2, and a VH CDR3 comprising the sequences of SEQ ID NOs: 9, 2, and 4, respectively. In some embodiments, the single domain antibody comprises a heavy chain variable (VH) region comprising a VH CDR1, a VH CDR2, and a VH CDR3 comprising the sequences of SEQ ID NOs: 1, 10, and 4, respectively.

In some embodiments, the single domain antibody comprises a heavy chain variable (VH) region comprising the CDR1, CDR2, and CDR3 of any one of SEQ ID NOs: 11-19. In some embodiments, the single domain antibody comprises a heavy chain variable (VH) region comprising the CDR1, CDR2, and CDR3 of SEQ ID NO: 11. In some embodiments, In some embodiments, the single domain antibody comprises a heavy chain variable (VH) region comprising the CDR1, CDR2, and CDR3 of SEQ ID NO: 12. In some embodiments, the single domain antibody comprises a heavy chain variable (VH) region comprising the CDR1, CDR2, and CDR3 of SEQ ID NO: 13. In some embodiments, the single domain antibody comprises a heavy chain variable (VH) region comprising the CDR1, CDR2, and CDR3 of SEQ ID NO: 14. In some embodiments, the single domain antibody comprises a heavy chain variable (VH) region comprising the CDR1, CDR2, and CDR3 of SEQ ID NO: 15. In some embodiments, the single domain antibody comprises a heavy chain variable (VH) region comprising the CDR1, CDR2, and CDR3 of SEQ ID NO: 16. In some embodiments, the single domain antibody comprises a heavy chain variable (VH) region comprising the CDR1, CDR2, and CDR3 of SEQ ID NO: 17. In some embodiments, the single domain antibody comprises a heavy chain variable (VH) region comprising the CDR1, CDR2, and CDR3 of SEQ ID NO: 18. In some embodiments, the single domain antibody comprises a heavy chain variable (VH) region comprising the CDR1, CDR2, and CDR3 of SEQ ID NO: 19.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, at least 85%, at least 90%, at least 95%) sequence identity to any one of SEQ ID NOs: 11-19. In some embodiments, the single domain antibody comprises a heavy chain variable (VH) region having at least 85% (such as, e.g., 85%, 90%, 95%, at least 90%, at least 95%) sequence identity to any one of SEQ ID NOs: 11-19. In some embodiments, the single domain antibody comprises a heavy chain variable (VH) region having at least 90% (such as, e.g., 90%, 95%, at least 95%) sequence identity to any one of SEQ ID NOs: 11-19. In some embodiments, the single domain antibody comprises a heavy chain variable (VH) region having at least 95% sequence identity to any one of SEQ ID NOs: 11-19.

In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 11. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 12. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 13. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 14. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 15. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 16. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 17. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 18. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 19.

In some embodiments, the single domain antibody comprises a heavy chain variable (VH) region chosen from SEQ ID NOs: 11-19.

In some embodiments, the single domain antibody comprises the heavy chain variable (VH) region of SEQ ID NO: 11. In some embodiments, the single domain antibody comprises the heavy chain variable (VH) region of SEQ ID NO: 12. In some embodiments, the single domain antibody comprises the heavy chain variable (VH) region of SEQ ID NO: 13. In some embodiments, the single domain antibody comprises the heavy chain variable (VH) region of SEQ ID NO: 14. In some embodiments, the single domain antibody comprises the heavy chain variable (VH) region of SEQ ID NO: 15. In some embodiments, the single domain antibody comprises the heavy chain variable (VH) region of SEQ ID NO: 16. In some embodiments, the single domain antibody comprises the heavy chain variable (VH) region of SEQ ID NO: 17. In some embodiments, the single domain antibody comprises the heavy chain variable (VH) region of SEQ ID NO: 18. In some embodiments, the single domain antibody comprises the heavy chain variable (VH) region of SEQ ID NO: 19.

In some embodiments, the single domain antibody specifically binds to human MSLN.

In some embodiments, the single domain antibody binds to human MSLN with a K_{D} of from about 10⁻⁹ M to about 10⁻⁶ M. In some embodiments, the single domain antibody binds to human MSLN with a K_{D} of ≤ 5 × 10⁻⁷ M. In some embodiments, the single domain antibody binds to human MSLN with a K_{D} of ≤ 1 × 10⁻⁷ M. In some embodiments, the single domain antibody binds to human MSLN with a K_{D} of ≤ 5 × 10⁻⁸ M. In some embodiments, the single domain antibody binds to human MSLN with a K_{D} of ≤ 2 × 10⁻⁸ M. In some embodiments, the single domain antibody binds to human MSLN with a K_{D} of ≤ 1 × 10⁻⁸ M. In some embodiments, the single domain antibody binds to human MSLN with a K_{D} of ≤ 1 × 10⁻⁹ M.

In some embodiments, the single domain antibody is a human single domain antibody.

In some embodiments, the single domain antibody is an isolated single domain antibody. In some embodiments, the single domain antibody is an isolated, human single domain antibody.

Some embodiments of the present disclosure relate to a mesothelin binding protein comprising a single domain antibody that specifically binds to mesothelin, as described herein.

In some embodiments, the single domain antibody comprises a heavy chain variable (VH) region comprising:
(i) a VH complementarity determining region one (CDR1) comprising the sequence G G S I S X₁ S Y Y (SEQ ID NO: 53), wherein X₁ is N or S;
(ii) a VH CDR2 comprising the sequence I Y X₂ S G X₃ X₄ (SEQ ID NO: 68), wherein X₂ is H or Y; X₃ is N or S; and X₄ is T or I; and
(iii) a VH CDR3 comprising the sequence X₅ X₆ Q X₇ G VG A T T TEE Y (SEQ ID NO: 54), wherein X₅ is T, V, or A; X₆ is S or T; and X₇ is D or N.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 85% (such as, e.g., 85%, 90%, 95%, at least 90%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 90% (such as, e.g., 90%, 95%, at least 95%) sequence identity to the CDRs 1, 2, and 3 of any one of SEQ ID NOs: 11-19. In some embodiments, the single domain antibody comprises a heavy chain variable (VH) region in which the full set of VH CDRs 1, 2, and 3 (combined) has at least 95% sequence identity to the CDRs 1, 2, and 3 of any one of SEQ ID NOs: 11-19.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 11. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 12. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 13. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 14. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 15. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 16. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 17. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 18. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the CDRs 1, 2, and 3 of SEQ ID NO: 19.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1 or SEQ ID NO: 9;
(ii) a VH CDR2 comprising a sequence having at most two (e.g., one, two, zero) amino acid modifications relative to SEQ ID NO: 2, SEQ ID NO: 7, or SEQ ID NO: 10; and
(iii) a VH CDR3 comprising a sequence having at most two (e.g., one, two, zero) amino acid modifications relative to SEQ ID NO: 3, SEQ ID NO: 4, SEQ ID NO: 5, SEQ ID NO: 6, or SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 3.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 4.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 5.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 6.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 7; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 9; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 6.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 9; (ii) a VH CDR2 comprising a sequence having at most two (e.g., one, two, zero) amino acid modifications relative to SEQ ID NO: 2; and (iii) a VH CDR3 comprising a sequence having at most two (e.g., one, two, zero) amino acid modifications relative to SEQ ID NO: 4.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 10; and (iii) a VH CDR3 comprising a sequence having at most two (e.g., one, two, zero) amino acid modifications relative to SEQ ID NO: 4.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1 or SEQ ID NO: 9. In some embodiments, the VH CDR2 comprises a sequence having at most one amino acid modification relative to SEQ ID NO: 2, SEQ ID NO: 7, or SEQ ID NO: 10. In some embodiments, the VH CDR3 comprises a sequence having at most one amino acid modification relative to SEQ ID NO: 3, SEQ ID NO: 4, SEQ ID NO: 5, SEQ ID NO: 6, or SEQ ID NO: 8. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1 and SEQ ID NO: 9. In some embodiments, the VH CDR2 comprises a sequence chosen from SEQ ID NO: 2, SEQ ID NO: 7, and SEQ ID NO: 10. In some embodiments, the VH CDR3 comprises a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(i) a VH complementarity determining region one (CDR1) comprising a sequence chosen from SEQ ID NO: 1 and SEQ ID NO: 9;
(ii) a VH CDR2 comprising a sequence chosen from SEQ ID NO: 2, SEQ ID NO: 7, and SEQ ID NO: 10; and
(iii) a VH CDR3 comprising a sequence chosen from SEQ ID NO: 3, SEQ ID NO: 4, SEQ ID NO: 5, SEQ ID NO: 6, and SEQ ID NO: 8.

In some embodiments, the single domain antibody comprises a heavy chain variable (VH) region comprising:
(a) a VH CDR1, a VH CDR2, and a VH CDR3 comprising the sequences of SEQ ID NOs: 1, 2, and 3, respectively;
(b) a VH CDR1, a VH CDR2, and a VH CDR3 comprising the sequences of SEQ ID NOs: 1, 2, and 4, respectively;
(c) a VH CDR1, a VH CDR2, and a VH CDR3 comprising the sequences of SEQ ID NOs: 1, 2, and 5, respectively;
(d) a VH CDR1, a VH CDR2, and a VH CDR3 comprising the sequences of SEQ ID NOs: 1, 2, and 6, respectively;
(e) a VH CDR1, a VH CDR2, and a VH CDR3 comprising the sequences of SEQ ID NOs: 1, 7, and 8, respectively;
(f) a VH CDR1, a VH CDR2, and a VH CDR3 comprising the sequences of SEQ ID NOs: 9, 2, and 6, respectively;
(g) a VH CDR1, a VH CDR2, and a VH CDR3 comprising the sequences of SEQ ID NOs: 9, 2, and 4, respectively; or
(h) a VH CDR1, a VH CDR2, and a VH CDR3 comprising the sequences of SEQ ID NOs: 1, 10, and 4, respectively.

In some embodiments, the single domain antibody comprises a heavy chain variable (VH) region comprising a VH CDR1, a VH CDR2, and a VH CDR3 comprising the sequences of SEQ ID NOs: 1, 2, and 3, respectively. In some embodiments, the single domain antibody comprises a heavy chain variable (VH) region comprising a VH CDR1, a VH CDR2, and a VH CDR3 comprising the sequences of SEQ ID NOs: 1, 2, and 4, respectively. In some embodiments, the single domain antibody comprises a heavy chain variable (VH) region comprising a VH CDR1, a VH CDR2, and a VH CDR3 comprising the sequences of SEQ ID NOs: 1, 2, and 5, respectively. In some embodiments, the single domain antibody comprises a heavy chain variable (VH) region comprising a VH CDR1, a VH CDR2, and a VH CDR3 comprising the sequences of SEQ ID NOs: 1, 2, and 6, respectively. In some embodiments, the single domain antibody comprises a heavy chain variable (VH) region comprising a VH CDR1, a VH CDR2, and a VH CDR3 comprising the sequences of SEQ ID NOs: 1, 7, and 8, respectively. In some embodiments, the single domain antibody comprises a heavy chain variable (VH) region comprising a VH CDR1, a VH CDR2, and a VH CDR3 comprising the sequences of SEQ ID NOs: 9, 2, and 6, respectively. In some embodiments, the single domain antibody comprises a heavy chain variable (VH) region comprising a VH CDR1, a VH CDR2, and a VH CDR3 comprising the sequences of SEQ ID NOs: 9, 2, and 4, respectively. In some embodiments, the single domain antibody comprises a heavy chain variable (VH) region comprising a VH CDR1, a VH CDR2, and a VH CDR3 comprising the sequences of SEQ ID NOs: 1, 10, and 4, respectively.

In some embodiments, the single domain antibody comprises a heavy chain variable (VH) region comprising the CDR1, CDR2, and CDR3 of any one of SEQ ID NOs: 11-19. In some embodiments, the single domain antibody comprises a heavy chain variable (VH) region comprising the CDR1, CDR2, and CDR3 of SEQ ID NO: 11. In some embodiments, In some embodiments, the single domain antibody comprises a heavy chain variable (VH) region comprising the CDR1, CDR2, and CDR3 of SEQ ID NO: 12. In some embodiments, the single domain antibody comprises a heavy chain variable (VH) region comprising the CDR1, CDR2, and CDR3 of SEQ ID NO: 13. In some embodiments, the single domain antibody comprises a heavy chain variable (VH) region comprising the CDR1, CDR2, and CDR3 of SEQ ID NO: 14. In some embodiments, the single domain antibody comprises a heavy chain variable (VH) region comprising the CDR1, CDR2, and CDR3 of SEQ ID NO: 15. In some embodiments, the single domain antibody comprises a heavy chain variable (VH) region comprising the CDR1, CDR2, and CDR3 of SEQ ID NO: 16. In some embodiments, the single domain antibody comprises a heavy chain variable (VH) region comprising the CDR1, CDR2, and CDR3 of SEQ ID NO: 17. In some embodiments, the single domain antibody comprises a heavy chain variable (VH) region comprising the CDR1, CDR2, and CDR3 of SEQ ID NO: 18. In some embodiments, the single domain antibody comprises a heavy chain variable (VH) region comprising the CDR1, CDR2, and CDR3 of SEQ ID NO: 19.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, at least 85%, at least 90%, at least 95%) sequence identity to any one of SEQ ID NOs: 11-19. In some embodiments, the single domain antibody comprises a heavy chain variable (VH) region having at least 85% (such as, e.g., 85%, 90%, 95%, at least 90%, at least 95%) sequence identity to any one of SEQ ID NOs: 11-19. In some embodiments, the single domain antibody comprises a heavy chain variable (VH) region having at least 90% (such as, e.g., 90%, 95%, at least 95%) sequence identity to any one of SEQ ID NOs: 11-19. In some embodiments, the single domain antibody comprises a heavy chain variable (VH) region having at least 95% sequence identity to any one of SEQ ID NOs: 11-19.

In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 11. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 12. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 13. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 14. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 15. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 16. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 17. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 18. In some embodiments, the single domain antibody comprises a heavy chain variable (VH) region having at least 80% (such as, e.g., 80%, 85%, 90%, 95%, at least 85%, at least 90%, at least 95%) sequence identity to SEQ ID NO: 19.

In some embodiments, the single domain antibody comprises a heavy chain variable (VH) region chosen from SEQ ID NOs: 11-19.

In some embodiments, the single domain antibody comprises the heavy chain variable (VH) region of SEQ ID NO: 11. In some embodiments, the single domain antibody comprises the heavy chain variable (VH) region of SEQ ID NO: 12. In some embodiments, the single domain antibody comprises the heavy chain variable (VH) region of SEQ ID NO: 13. In some embodiments, the single domain antibody comprises the heavy chain variable (VH) region of SEQ ID NO: 14. In some embodiments, the single domain antibody comprises the heavy chain variable (VH) region of SEQ ID NO: 15. In some embodiments, the single domain antibody comprises the heavy chain variable (VH) region of SEQ ID NO: 16. In some embodiments, the single domain antibody comprises the heavy chain variable (VH) region of SEQ ID NO: 17. In some embodiments, the single domain antibody comprises the heavy chain variable (VH) region of SEQ ID NO: 18. In some embodiments, the single domain antibody comprises the heavy chain variable (VH) region of SEQ ID NO: 19.

In some embodiments, the mesothelin binding protein specifically binds to human MSLN.

In some embodiments, the mesothelin binding protein binds to human MSLN with a K_{D} of from about 10⁻⁹ M to about 10⁻⁶ M. In some embodiments, the mesothelin binding protein binds to human MSLN with a K_{D} of ≤ 5 × 10⁻⁷ M. In some embodiments, the mesothelin binding protein binds to human MSLN with a K_{D} of ≤ 1 × 10⁻⁷ M. In some embodiments, the mesothelin binding protein binds to human MSLN with a K_{D} of ≤ 5 × 10⁻⁸ M. In some embodiments, the mesothelin binding protein binds to human MSLN with a K_{D} of ≤ 2 × 10⁻⁸ M. In some embodiments, the mesothelin binding protein binds to human MSLN with a K_{D} of ≤ 1 × 10⁻⁸ M. In some embodiments, the mesothelin binding protein binds to human MSLN with a K_{D} of ≤ 1 × 10⁻⁹ M.

In some embodiments, the mesothelin binding protein further binds to one or more target antigens other than mesothelin. In some embodiments, the mesothelin binding protein is multispecific. In some embodiments, the mesothelin binding protein is bispecific.

In some embodiments, the mesothelin binding protein further specifically binds to CD3. In some embodiments, the mesothelin binding protein further specifically binds to human CD3. In some embodiments, the mesothelin binding protein binds to human CD3 with a K_{D} of from about 10⁻⁹ M to about 10⁻⁶ M. In some embodiments, the mesothelin binding protein binds to human CD3 with a K_{D} of ≤ 5 × 10⁻⁷ M. In some embodiments, the mesothelin binding protein binds to human CD3 with a K_{D} of ≤ 1 × 10⁻⁷ M. In some embodiments, the mesothelin binding protein binds to human CD3 with a K_{D} of ≤ 5 × 10⁻⁸ M. In some embodiments, the mesothelin binding protein binds to human CD3 with a K_{D} of ≤ 2 × 10⁻⁸ M. In some embodiments, the mesothelin binding protein binds to human CD3 with a K_{D} of ≤ 1 × 10⁻⁸ M. In some embodiments, the mesothelin binding protein binds to human CD3 with a K_{D} of ≤ 1 × 10⁻⁹ M.

In some embodiments, the mesothelin binding protein binds to human MSLN and/or CD3 with a K_{D} of from about 10⁻⁹ M to about 10⁻⁶ M. In some embodiments, the mesothelin binding protein binds to human MSLN and/or CD3 with a K_{D} of ≤ 5 × 10⁻⁷ M. In some embodiments, the mesothelin binding protein binds to human MSLN and/or CD3 with a K_{D} of ≤ 1 × 10⁻⁷ M. In some embodiments, the mesothelin binding protein binds to human MSLN and/or CD3 with a K_{D} of ≤ 5 × 10⁻⁸ M. In some embodiments, the mesothelin binding protein binds to human MSLN and/or CD3 with a K_{D} of ≤ 2 × 10⁻⁸ M. In some embodiments, the mesothelin binding protein binds to human MSLN and/or CD3 with a K_{D} of ≤ 1 × 10⁻⁸ M. In some embodiments, the mesothelin binding protein binds to human MSLN and/or CD3 with a K_{D} of ≤ 1 × 10⁻⁹ M.

In some embodiments, the mesothelin binding protein further specifically binds to human CD3 epsilon. In some embodiments, the mesothelin binding protein binds to an epitope on CD3 comprising at least one residue selected from CD3 epsilon (SEQ ID NO: 69): K73 and S83; and CD3 delta (SEQ ID NO: 70) K82 and C93. In some embodiments, the epitope on CD3 comprises the region of CD3 delta defined by K82, E83, S84, T85, V86, Q87, V88, H89, Y90, R91, M92, C93. In some embodiments, the epitope on CD3 comprises the region of CD3 epsilon defined by K73, N74, 175, G76, S77, D78, E79, D80, H81, L82, S83. In some embodiments, the epitope comprises a conformational epitope with residues of both CD3 delta and CD3 epsilon. In some embodiments, the conformational epitope comprises each of residues CD3ε K73 and S83; CD3δ K82 and C93.

In some embodiments, the mesothelin binding protein further comprises a CD3-binding VH region. In some embodiments, the mesothelin binding protein further comprises a CD3-binding VH region that is paired with a light chain (LV) region.

In some embodiments, the CD3-binding VH region may belong to a family of closely related single domain antibodies that specifically bind to human CD3. The single domain antibodies of this family comprise a set of CDR sequences as defined herein and as shown in Tables S4 and S5, and are exemplified by the provided heavy chain variable region (VH) sequences of SEQ ID NOs: 31-48 as set forth in Table S6. Multi-specific molecules comprising these CD3-binding VH domains and their associated light chain variable domain (as described in Tables S7 and S8) have advantageous properties, for example, as described in published PCT application publication number WO2018/052503, the disclosure of which is incorporated by reference herein in its entirety. Any of the single domain antibodies described herein that specifically binds to MSLN can be combined with the CD3-binding domains and fixed light chain domains described herein to generate a multispecific mesothelin binding protein.

TABLE S4

Anti-CD3 Heavy Chain Antibody CDR1, CDR2 and CDR3 Amino Acid Sequences

| Clone ID # | SEQ_aa_CDR1 | SEQ_aa_CDR2 | SEQ_aa_CDR3 |
|---|---|---|---|
| 312557 | GFTFDDYA (SEQ ID NO: 20) | ISWNSGSI (SEQ ID NO: 26) | AKDSRGYGDYRLGGAY (SEQ ID NO: 27) |
| 308261 | GFTFDDYA (SEQ ID NO: 20) | ISWNSGSI (SEQ ID NO: 26) | AKDSRGYGDYRLGGAY (SEQ ID NO: 27) |
| 308159 | GFTFDDYA (SEQ ID NO: 20) | ISWNSGSI (SEQ ID NO: 26) | AKDSRGYGDYSRGGAY (SEQ ID NO: 28) |
| 308160 | GFTFDDYA (SEQ ID NO: 20) | ISWNSGSI (SEQ ID NO: 26) | AKDSRGYGDYSLGGAY (SEQ ID NO: 29) |
| 308256 | GFTFDDYA (SEQ ID NO: 20) | ISWNSGSI (SEQ ID NO: 26) | AKDSRGYGDYSLGGAY (SEQ ID NO: 29) |
| 312585 | GFTFANYA (SEQ ID NO: 21) | ISWNSGSI (SEQ ID NO: 26) | AKDSRGYGDYSRGGAY (SEQ ID NO: 28) |
| 312614 | GFTFNNYA (SEQ ID NO: 22) | ISWNSGSI (SEQ ID NO: 26) | AKDSRGYGDYSRGGAY (SEQ ID NO: 28) |
| 312583 | GFTFADYA (SEQ ID NO: 23) | ISWNSGSI (SEQ ID NO: 26) | AKDSRGYGDYSRGGAY (SEQ ID NO: 28) |
| 312586 | GFTFDNYA (SEQ ID NO: 24) | ISWNSGSI (SEQ ID NO: 26) | AKDSRGYGDYSRGGAY (SEQ ID NO: 28) |
| 312624 | GFTFDNYA (SEQ ID NO: 24) | ISWNSGSI (SEQ ID NO: 26) | AKDSRGYGDYSRGGAY (SEQ ID NO: 28) |
| 312578 | GFTFDDYA (SEQ ID NO: 20) | ISWNSGSI (SEQ ID NO: 26) | AKDSRGYGDYSRGGAY (SEQ ID NO: 28) |
| 312620 | GFTFDDYA (SEQ ID NO: 20) | ISWNSGSI (SEQ ID NO: 26) | AKDSRGYGSYSRGGAY (SEQ ID NO: 30) |
| 312634 | GFTFHNYA (SEQ ID NO: 25) | ISWNSGSI (SEQ ID NO: 26) | AKDSRGYGDYSLGGAY (SEQ ID NO: 29) |
| 312579 | GFTFDDYA (SEQ ID NO: 20) | ISWNSGSI (SEQ ID NO: 26) | AKDSRGYGDYSRGGAY (SEQ ID NO: 28) |
| 312630 | GFTFDNYA (SEQ ID NO: 24) | ISWNSGSI (SEQ ID NO: 26) | AKDSRGYGDYSLGGAY (SEQ ID NO: 29) |
| 312570 | GFTFDDYA (SEQ ID NO: 20) | ISWNSGSI (SEQ ID NO: 26) | AKDSRGYGDYRLGGAY (SEQ ID NO: 27) |
| 312567 | GFTFDDYA (SEQ ID NO: 20) | ISWNSGSI (SEQ ID NO: 26) | AKDSRGYGDYRLGGAY (SEQ ID NO: 27) |
| 312558 | GFTFDDYA (SEQ ID NO: 20) | ISWNSGSI (SEQ ID NO: 26) | AKDSRGYGDYRLGGAY (SEQ ID NO: 27) |

TABLE S5

Anti-CD3 Heavy Chain Antibody Unique CDR Amino Acid Sequences

| | | |
|---|---|---|
| SEQ_aa_CDR1 | SEQ_aa_CDR2 | SEQ_aa_CDR3 |
| GFTFDDYA (SEQ ID NO: 20) | ISWNSGSI (SEQ ID NO: 26) | AKDSRGYGDYRLGGAY (SEQ ID NO: 27) |
| GFTFANYA (SEQ ID NO: 21) | | AKDSRGYGDYSRGGAY (SEQ ID NO: 28) |
| GFTFNNYA (SEQ ID NO: 22) | | AKDSRGYGDYSLGGAY (SEQ ID NO: 29) |
| GFTFADYA (SEQ ID NO: 23) | | AKDSRGYGSYSRGGAY (SEQ ID NO: 30) |
| GFTFDNYA (SEQ ID NO: 24) | | |
| GFTFHNYA (SEQ ID NO: 25) | | |

TABLE S6

Anti-CD3 Heavy Chain Antibody Variable Domain Amino Acid Sequences

| Clone ID # | SEQ_aa_FR1_FR4 | SEQ ID NO. |
|---|---|---|
| 312557 | EVQLVESGGGLVQPGRSLRLSCAASGFTFDDYAMHWVRQAP GKGLEWVSGISWNSGSIGYADSVKGRFTISRDNAKNSLYLQM NSLRAEDTALYYCAKDSRGYGDYRLGGAYWGQGTLVTVSS | 31 |
| 308261 | EVQLVESGGGLVKPGGSLRLSCAASGFTFDDYAMHWVRQAP GKGLEWVSGISWNSGSIGYADSVKGRFTISRDNAKNSLYLQM NSLRAEDTALYYCAKDSRGYGDYRLGGAYWGQGTLVTVSS | 32 |
| 308159 | EVQLVESGGGLVQPGRSLRLSCAASGFTFDDYAMHWVRQAP GKGLEWVSGISWNSGSIGYADSVKGRFTISRDNAKKSLYLQM NSLRAEDTALYYCAKDSRGYGDYSRGGAYWGQGTLVTVSS | 33 |
| 308160 | EVQLVESGGGLVQPGRSLRLSCAASGFTFDDYAMHWVRQAP GKGLEWVSGISWNSGSIGYADSVKGRFTISRDNAKNSLYLQM NSLRPEDTALYYCAKDSRGYGDYSLGGAYWGQGTLVTVSS | 34 |
| 308256 | EVQLVESGGGLVQPGRSLRLSCAASGFTFDDYAMHWVRQAP GKGLEWVSGISWNSGSIGYADSVKGRFTISRDNAKNSLYLQM NSLRAEDTALYYCAKDSRGYGDYSLGGAYWGQGTLVTVSS | 35 |
| 312585 | EVQLVESGGGLVQPGRSLRLSCAASGFTFANYAMHWVRQAP GKGLEWVSGISWNSGSIDYADSVKGRFTISRDNAKNSLYLQM NSLRAEDTALYYCAKDSRGYGDYSRGGAYWGQGTLVTVSS | 36 |
| 312614 | EVQLVESGGGLVQPGRSLRLSCAASGFTFNNYAMHWVRQAP GKGLEWVSGISWNSGSIGYADSVKGRFTISRDNAKNSLYLQM NSLRGEDTALYYCAKDSRGYGDYSRGGAYWGQGTLVTVSS | 37 |
| 312583 | EVQLVESGGGLVQPGGSLRLSCAASGFTFADYAMHWVRQAP GKGLEWVSGISWNSGSIGYADSVKGRFTISRDNAKNSLYLQM NSLRAEDTALYYCAKDSRGYGDYSRGGAYWGQGTLVTVSS | 38 |
| 312586 | EVQLVESGGGLVQPGRSLRLSCAASGFTFDNYAMHWVRQAP GKGLEWVSGISWNSGSIGYADSVKGRFTISRDNAKKSLYLQM NSLRAEDTALYYCAKDSRGYGDYSRGGAYWGQGTLVTVSS | 39 |
| 312624 | EVQLVESGGGLVQPGRSLRLSCAASGFTFDNYAMHWVRQAP GKGLEWVSGISWNSGSIGYADSVKGRFTISRDNAKNSLYLQM NSLRAEDTALYYCAKDSRGYGDYSRGGAYWGQGTLVTVSS | 40 |
| 312578 | EVQLVESGGGLVQPGRSLRLSCAASGFTFDDYAMHWVRQAP GKGLEWVSGISWNSGSIGYADSVKGRFTISRDNAKNSLYLQM NSLRAEDTALYYCAKDSRGYGDYSRGGAYWGQGTLVTVSS | 41 |
| 312620 | EVQLVESGGGLVQPGRSLRLSCAASGFTFDDYAMHWVRQAP GKGLEWVSGISWNSGSIGYADSVKGRFTISRDNAKNSLYLQM NSLRPEDTALYYCAKDSRGYGSYSRGGAYWGQGTLVTVSS | 42 |
| 312634 | EVQLVESGGGLVQPGRSLRLSCAASGFTFHNYAMHWVRQAP GKGLEWVSGISWNSGSIGYADSVKGRFTISRDNAKNSLYLQM NSLRAEDTALYYCAKDSRGYGDYSLGGAYWGQGTLVTVSS | 43 |
| 312579 | EVQLVESGGGLVQPGGSLRLSCAASGFTFDDYAMHWVRQAP GKGLEWVSGISWNSGSIGYADSVKGRFTISRDNAKNSLYLQM NSLRAEDTALYYCAKDSRGYGDYSRGGAYCGQGTLVTVSS | 44 |
| 312630 | EVQLVESGGGLVQPGRSLRLSCAASGFTFDNYAMHWVRQAP GKGLEWVSGISWNSGSIGYADSVKGRFTISRDNAKKSLYLQM NSLRAEDTALYYCAKDSRGYGDYSLGGAYWGQGTLVTVSS | 45 |
| 312570 | EVQLVESGGGLVQPGRSLRLSCAASGFTFDDYAMHWVRQAP GKGLEWVSGISWNSGSIGYADSVKGRFTISRDNAKKSLYLQM NSLRAEDTALYYCAKDSRGYGDYRLGGAYWGQGTLVTVSS | 46 |
| 312567 | EVQLVESGGGLVQPGRSLRLSCEASGFTFDDYAMHWVRQAP GKGLEWVSGISWNSGSIGYADSVKGRFTISRDNAKNSLYLQM NSLRAEDTALYYCAKDSRGYGDYRLGGAYWGQGTLVTVSS | 47 |
| 312558 | EVQLVESGGGLVQPGRSLRLSCAASGFTFDDYAMHWVRQAP GKGLEWVSGISWNSGSIGYADSVKGRFTISRDNAKNSLYLQM NSLRPEDTALYYCAKDSRGYGDYRLGGAYWGQGTLVTVSS | 48 |

TABLE S7

Anti-CD3 Arm Fixed Light Chain Antibody CDR1, CDR2 and CDR3 Amino Acid Sequences

| | | |
|---|---|---|
| SEQ_aa_CDR1 | SEQ_aa_CDR2 | SEQ_aa_CDR3 |
| QSVSSN (SEQ ID NO: 49) | GAS (SEQ ID NO: 50) | QQYNNWPWT (SEQ ID NO: 51) |

TABLE S8

Anti-CD3 Arm Fixed Light Chain Antibody Variable Domain Amino Acid Sequence

| Clone ID # | SEQ_aa_FR1_FR4 | SEQ ID NO. |
|---|---|---|
| 312325 | EIVMTQSPATLSVSPGERATLSCRASQSVSSNLAWYQQKPGQ APRLLIYGASTRATGIPARFSGSGSGTEFTLTISSLQSEDFAVYY CQQYNNWPWTFGQGTKVEIK | 52 |

In some embodiments, the CD3-binding VH region comprises:
(i) a VH complementarity determining region one (CDR1) comprising a sequence having at most two (such as, e.g., zero, one, or two) amino acid modifications relative to any one of SEQ ID NOs: 20-25;
(ii) a VH CDR2 comprising a sequence having at most two (such as, e.g., zero, one, or two) amino acid modifications relative to SEQ ID NO: 26; and
(iii) a VH CDR3 comprising a sequence having at most two (such as, e.g., zero, one, or two) amino acid modifications relative to any one of SEQ ID NOs: 27-30.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 27. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 21; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 22; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 23; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 24; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 30. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 25; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 24; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, each amino acid modification, if any, is a conservative amino acid substitution. In some embodiments, each amino acid modification, if any, is a conservative amino acid substitution listed in Table A1.

In some embodiments, the CD3-binding VH CDR1 comprises a sequence having at most one amino acid modification relative to any one of SEQ ID NO: 20-25. In some embodiments, the CD3-binding VH CDR2 comprises a sequence having at most one amino acid modification relative to SEQ ID NO: 26. In some embodiments, the CD3-binding VH CDR3 comprises a sequence having at most one amino acid modification relative to any one of SEQ ID NOs: 27-30. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the CD3-binding VH CDR1 comprises a sequence chosen from SEQ ID NOs: 20-25. In some embodiments, the CD3-binding VH CDR1 comprises the sequence of SEQ ID NO: 20. In some embodiments, the CD3-binding VH CDR1 comprises the sequence of SEQ ID NO: 21. In some embodiments, the CD3-binding VH CDR1 comprises the sequence of SEQ ID NO: 22. In some embodiments, the CD3-binding VH CDR1 comprises the sequence of SEQ ID NO: 23. In some embodiments, the CD3-binding VH CDR1 comprises the sequence of SEQ ID NO: 24. In some embodiments, the CD3-binding VH CDR1 comprises the sequence of SEQ ID NO: 25.

In some embodiments, the CD3-binding VH CDR2 comprises the sequence of SEQ ID NO: 26.

In some embodiments, the CD3-binding VH CDR3 comprises a sequence chosen from SEQ ID NOs: 27-30. In some embodiments, the CD3-binding VH CDR3 comprises the sequence of SEQ ID NO: 27. In some embodiments, the CD3-binding VH CDR3 comprises the sequence of SEQ ID NO: 28. In some embodiments, the CD3-binding VH CDR3 comprises the sequence of SEQ ID NO: 29. In some embodiments, the CD3-binding VH CDR3 comprises the sequence of SEQ ID NO: 30.

In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 85% (such as, e.g., 85%, 90%, 95%, at least 90%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 90% (such as, e.g., 90%, 95%, at least 95%) sequence identity to the CDRs 1, 2, and 3 of any one of SEQ ID NOs: 31-48. In some embodiments, the full set of VH CDRs 1, 2, and 3 (combined) in the CD3-binding VH region has at least 95% sequence identity to the CDRs 1, 2, and 3 of any one of SEQ ID NOs: 31-48.

In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 31. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 32. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 33. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 34. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 35. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 36. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 37. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 38. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 39. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 40. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 41. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 42. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 43. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 44. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 45. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 46. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 47. In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the CDRs 1, 2, and 3 of SEQ ID NO: 48.

In some embodiments, the CD3-binding VH region comprises the CDR1, CDR2, and CDR3 of any one of SEQ ID NOs: 31-48. In some embodiments, the CD3-binding VH region comprises the CDR1, CDR2, and CDR3 of SEQ ID NO: 31. In some embodiments, the CD3-binding VH region comprises the CDR1, CDR2, and CDR3 of SEQ ID NO: 32. In some embodiments, the CD3-binding VH region comprises the CDR1, CDR2, and CDR3 of SEQ ID NO: 33. In some embodiments, the CD3-binding VH region comprises the CDR1, CDR2, and CDR3 of SEQ ID NO: 34. In some embodiments, the CD3-binding VH region comprises the CDR1, CDR2, and CDR3 of SEQ ID NO: 35. In some embodiments, the CD3-binding VH region comprises the CDR1, CDR2, and CDR3 of SEQ ID NO: 36. In some embodiments, the CD3-binding VH region comprises the CDR1, CDR2, and CDR3 of SEQ ID NO: 37. In some embodiments, the CD3-binding VH region comprises the CDR1, CDR2, and CDR3 of SEQ ID NO: 38. In some embodiments, the CD3-binding VH region comprises the CDR1, CDR2, and CDR3 of SEQ ID NO: 39. In some embodiments, the CD3-binding VH region comprises the CDR1, CDR2, and CDR3 of SEQ ID NO: 40. In some embodiments, the CD3-binding VH region comprises the CDR1, CDR2, and CDR3 of SEQ ID NO: 41. In some embodiments, the CD3-binding VH region comprises the CDR1, CDR2, and CDR3 of SEQ ID NO: 42. In some embodiments, the CD3-binding VH region comprises the CDR1, CDR2, and CDR3 of SEQ ID NO: 43. In some embodiments, the CD3-binding VH region comprises the CDR1, CDR2, and CDR3 of SEQ ID NO: 44. In some embodiments, the CD3-binding VH region comprises the CDR1, CDR2, and CDR3 of SEQ ID NO: 45. In some embodiments, the CD3-binding VH region comprises the CDR1, CDR2, and CDR3 of SEQ ID NO: 46. In some embodiments, the CD3-binding VH region comprises the CDR1, CDR2, and CDR3 of SEQ ID NO: 47. In some embodiments, the CD3-binding VH region comprises the CDR1, CDR2, and CDR3 of SEQ ID NO: 48.

In some embodiments, the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

In some embodiments, the CD3-binding VH region comprises a VH CDR1, a VH CDR2, and a VH CDR3 comprising the sequences of SEQ ID NOs: 20, 26, and 27, respectively. In some embodiments, the CD3-binding VH region comprises a VH CDR1, a VH CDR2, and a VH CDR3 comprising the sequences of SEQ ID NOs: 20, 26, and 28, respectively. In some embodiments, the CD3-binding VH region comprises a VH CDR1, a VH CDR2, and a VH CDR3 comprising the sequences of SEQ ID NOs: 20, 26, and 29, respectively. In some embodiments, the CD3-binding VH region comprises a VH CDR1, a VH CDR2, and a VH CDR3 comprising the sequences of SEQ ID NOs: 21, 26, and 28, respectively. In some embodiments, the CD3-binding VH region comprises a VH CDR1, a VH CDR2, and a VH CDR3 comprising the sequences of SEQ ID NOs: 22, 26, and 28, respectively. In some embodiments, the CD3-binding VH region comprises a VH CDR1, a VH CDR2, and a VH CDR3 comprising the sequences of SEQ ID NOs: 23, 26, and 28, respectively. In some embodiments, the CD3-binding VH region comprises a VH CDR1, a VH CDR2, and a VH CDR3 comprising the sequences of SEQ ID NOs: 24, 26, and 28, respectively. In some embodiments, the CD3-binding VH region comprises a VH CDR1, a VH CDR2, and a VH CDR3 comprising the sequences of SEQ ID NOs: 20, 26, and 30, respectively. In some embodiments, the CD3-binding VH region comprises a VH CDR1, a VH CDR2, and a VH CDR3 comprising the sequences of SEQ ID NOs: 25, 26, and 29, respectively. In some embodiments, the CD3-binding VH region comprises a VH CDR1, a VH CDR2, and a VH CDR3 comprising the sequences of SEQ ID NOs: 24, 26, and 29, respectively.

In some embodiments, the CD3-binding VH region comprises:
(i) a VH complementarity determining region one (CDR1) comprising the sequence GF TF X₈ X₉ Y A (SEQ ID NO: 55), wherein X₈ is D, A, or H and X₉ is D or N;
(ii) a VH CDR2 comprising the sequence ISWNSGSI (SEQ ID NO: 26); and
(iii) a VH CDR3 comprising the sequence A K D S R G Y G X₁₀ Y X₁₁ X₁₂ G G A Y (SEQ ID NO: 56), wherein X₁₀ is D or S; X₁₁ is R or S; and X₁₂ is L or R.

In some embodiments, the VH CDR1, VH CDR2, and VH CDR3 sequences in the CD3-binding VH region are present in a human VH framework.

In some embodiments, the CD3-binding VH region has at least 80% (such as, e.g., 80%, 85%, 90%, 95%, at least 85%, at least 90%, at least 95%) sequence identity to any one of SEQ ID NOs: 31-48. In some embodiments, the CD3-binding VH region has at least 85% (such as, e.g., 85%, 90%, 95%, at least 90%, at least 95%) sequence identity to any one of SEQ ID NOs: 31-48. In some embodiments, the CD3-binding VH region has at least 90% (such as, e.g., 90%, 95%, at least 95%) sequence identity to any one of SEQ ID NOs: 31-48. In some embodiments, the CD3-binding VH region has at least 95% sequence identity to any one of SEQ ID NOs: 31-48.

In some embodiments, the CD3-binding VH region has at least 80% (such as, e.g., 80%, 85%, 90%, 95%, at least 85%, at least 90%, at least 95%) sequence identity to SEQ ID NO: 31. In some embodiments, the CD3-binding VH region has at least 80% (such as, e.g., 80%, 85%, 90%, 95%, at least 85%, at least 90%, at least 95%) sequence identity to SEQ ID NO: 32. In some embodiments, the CD3-binding VH region has at least 80% (such as, e.g., 80%, 85%, 90%, 95%, at least 85%, at least 90%, at least 95%) sequence identity to SEQ ID NO: 33. In some embodiments, the CD3-binding VH region has at least 80% (such as, e.g., 80%, 85%, 90%, 95%, at least 85%, at least 90%, at least 95%) sequence identity to SEQ ID NO: 34. In some embodiments, the CD3-binding VH region has at least 80% (such as, e.g., 80%, 85%, 90%, 95%, at least 85%, at least 90%, at least 95%) sequence identity to SEQ ID NO: 35. In some embodiments, the CD3-binding VH region has at least 80% (such as, e.g., 80%, 85%, 90%, 95%, at least 85%, at least 90%, at least 95%) sequence identity to SEQ ID NO: 36. In some embodiments, the CD3-binding VH region has at least 80% (such as, e.g., 80%, 85%, 90%, 95%, at least 85%, at least 90%, at least 95%) sequence identity to SEQ ID NO: 37. In some embodiments, the CD3-binding VH region has at least 80% (such as, e.g., 80%, 85%, 90%, 95%, at least 85%, at least 90%, at least 95%) sequence identity to SEQ ID NO: 38. In some embodiments, the CD3-binding VH region has at least 80% (such as, e.g., 80%, 85%, 90%, 95%, at least 85%, at least 90%, at least 95%) sequence identity to SEQ ID NO: 39. In some embodiments, the CD3-binding VH region has at least 80% (such as, e.g., 80%, 85%, 90%, 95%, at least 85%, at least 90%, at least 95%) sequence identity to SEQ ID NO: 40. In some embodiments, the CD3-binding VH region has at least 80% (such as, e.g., 80%, 85%, 90%, 95%, at least 85%, at least 90%, at least 95%) sequence identity to SEQ ID NO: 41. In some embodiments, the CD3-binding VH region has at least 80% (such as, e.g., 80%, 85%, 90%, 95%, at least 85%, at least 90%, at least 95%) sequence identity to SEQ ID NO: 42. In some embodiments, the CD3-binding VH region has at least 80% (such as, e.g., 80%, 85%, 90%, 95%, at least 85%, at least 90%, at least 95%) sequence identity to SEQ ID NO: 43. In some embodiments, the CD3-binding VH region has at least 80% (such as, e.g., 80%, 85%, 90%, 95%, at least 85%, at least 90%, at least 95%) sequence identity to SEQ ID NO: 44. In some embodiments, the CD3-binding VH region has at least 80% (such as, e.g., 80%, 85%, 90%, 95%, at least 85%, at least 90%, at least 95%) sequence identity to SEQ ID NO: 45. In some embodiments, the CD3-binding VH region has at least 80% (such as, e.g., 80%, 85%, 90%, 95%, at least 85%, at least 90%, at least 95%) sequence identity to SEQ ID NO: 46. In some embodiments, the CD3-binding VH region has at least 80% (such as, e.g., 80%, 85%, 90%, 95%, at least 85%, at least 90%, at least 95%) sequence identity to SEQ ID NO: 47. In some embodiments, the CD3-binding VH region has at least 80% (such as, e.g., 80%, 85%, 90%, 95%, at least 85%, at least 90%, at least 95%) sequence identity to SEQ ID NO: 48.

In some embodiments, the light chain variable region comprises the CDR1, CDR2, and CDR3 of SEQ ID NO: 52. In some embodiments, the light chain variable region comprises a VL CDR1, a VL CDR2, and a VL CDR3 comprising the sequence of SEQ ID NOs: 49, 50, and 51, respectively. In some embodiments, the VL CDR1, VL CDR2, and VL CDR3 sequences are present in a human VH framework.

In some embodiments, the light chain variable region has at least 80% (such as, e.g., 80%, 85%, 90%, 95%, at least 85%, at least 90%, at least 95%) sequence identity to SEQ ID NO: 52. In some embodiments, the light chain variable region has at least 85% (such as, e.g., 85%, 90%, 95%, at least 90%, at least 95%) sequence identity to SEQ ID NO: 52. In some embodiments, the light chain variable region has at least 90% (such as, e.g., 90%, 95%, at least 95%) sequence identity to SEQ ID NO: 52. In some embodiments, the light chain variable region has at least 95% sequence identity to SEQ ID NO: 52.

In some embodiments, the mesothelin binding protein is an anti-mesothelin antibody or fragment thereof.

In some embodiments, the anti-mesothelin antibody is a monoclonal antibody or fragment thereof. In some embodiments, the anti-mesothelin antibody is an isolated monoclonal antibody or fragment thereof.

In some embodiments, the anti-mesothelin antibody is an intact IgG molecule. In some embodiments, the anti-mesothelin antibody is an intact IgG1 molecule. In some embodiments, the anti-mesothelin antibody is an intact IgG2 molecule. In some embodiments, the anti-mesothelin antibody is an intact IgG4 molecule.

In some embodiments, the mesothelin binding protein is an antibody fragment. In some embodiments, the antibody fragment is an immunologically active portion of an intact IgG molecule. In some embodiments, the antibody fragment is an immunologically active portion of an intact IgG1 molecule. In some embodiments, the antibody fragment is an immunologically active portion of an intact IgG2 molecule. In some embodiments, the antibody fragment is an immunologically active portion of an intact IgG4 molecule. In some embodiments, the antibody fragment is a heavy chain-only antibody. In some embodiments, the antibody fragment is a TCA.

In some embodiments, the anti-mesothelin antibody or fragment thereof further comprises a Fc region. In some embodiments, the anti-mesothelin antibody or fragment thereof further comprises a variant Fc region. In some embodiments, the variant Fc region possesses at least about 80% homology (e.g., at least about 85%, at least about 90%, at least about 95%, at least about 98%, at least about 99%) with a native sequence Fc region.

In some embodiments, the variant Fc region comprises heterodimerizing alterations. In some embodiments, the heterodimerizing alterations comprise knob and holes substitutions (such as, e.g., in a variant IgG1 Fc region, 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; or 9) T366W in one polypeptide of the Fc and T366S, L368A, and Y407V in the other). In some embodiments, the heterodimerizing alterations comprise substitutions that create new disulfide bridges (such as, e.g., in a variant IgG1 Fc region, 1) Y349C in one Fc polypeptide chain and S354C in the other; 2) Y349C in one Fc polypeptide chain and E356C in the other; 3) Y349C in one Fc polypeptide chain and E357C in the other; 4) L351C in one Fc polypeptide chain and S354C in the other; 5) T394C in one Fc polypeptide chain and E397C in the other; or 6) D399C in one Fc polypeptide chain and K392C in the other). In some embodiments, the heterodimerizing alterations comprise charge pair substitutions (such as, e.g., 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; or 17) K409D and K439D on one chain plus D399K and E356K on the other).

In some embodiments, the Fc region is a silenced Fc region. In some embodiments, the silenced Fc region comprises substitution of one or more (such as, e.g., two or more) of Fc region residues 238, 265, 269, 270, 297, 327 and 329 according to EU numbering. In some embodiments, the silenced Fc region comprises a substitution that alters glycosylation. In some embodiments, the silenced Fc region comprises an effector-less mutation (such as, e.g., an N297A, an N297G, a DANA mutation (D265A+N297A), or a DANG mutation (D265A+N297G) in the CH2 region). In some embodiments, the silenced Fc region comprises K322A and L234A/L235A mutations.

In some embodiments, an anti-mesothelin antibody or fragment thereof further comprises a heavy chain constant region sequence in the absence of a CH1 sequence. In some embodiments, the anti-mesothelin antibody or fragment thereof comprises a heavy chain constant region comprising a hinge region, a CH2 domain, and a CH3 domain. In some embodiments, the hinge region comprises a wild type human IgG4 hinge region sequence (SEQ ID NO: 61). In some embodiments, the hinge region comprises a variant human IgG4 hinge region sequence comprising an S228P mutation (SEQ ID NO: 62). In some embodiments, the CH2 domain comprises a wild type human IgG4 CH2 domain sequence (SEQ ID NO: 63). In some embodiments, the CH2 domain comprises a variant human IgG4 CH2 domain comprising an F234A mutation, an L235A mutation, or both an F234A mutation and an L235A mutation. In some embodiments, the CH3 domain comprises a wild type human IgG4 CH3 domain sequence (SEQ ID NO: 65). In some embodiments, the CH3 domain comprises a variant human IgG4 CH3 domain sequence comprising a T366W mutation. In some embodiments, the CH3 domain comprises a variant human IgG4 CH3 domain sequence comprising a T366S, an L368A mutation, and a Y407V mutation.

Table S9 provides the sequences of human IgG1 and IgG4 Fc region sequences, as well as versions of these sequences that incorporate additional mutations (variants) that may impart additional desired properties to anti-mesothelin antibodies and fragments thereof described herein. Table S10 provides example human CD3 epsilon and CD3 delta sequences.

TABLE S9

Human IgG1 and IgG4 Fc Region Sequences and Variants Thereof

| | |
|---|---|
| Polypeptide Name | Amino Acid Sequence |
| Human IgG1 (UniProt No. P01857) | ASTKGPSVFPLAPSSKSTSGGTAALGCLVKDYFPEPVTVS WNSGALTSGVHTFPAVLQSSGLYSLSSVVTVPSSSLGTQT YICNVNHKPSNTKVDKKVEPKSCDKTHTCPPCPAPELLGG PSVFLFPPKPKDTLMISRTPEVTCVVVDVSHEDPEVKFNW YVDGVEVHNAKTKPREEQYNSTYRVVSVLTVLHQDWLNGK EYKCKVSNKALPAPIEKTISKAKGQPREPQVYTLPPSRDE LTKNQVSLTCLVKGFYPSDIAVEWESNGQPENNYKTTPPV LDSDGSFFLYSKLTVDKSRWQQGNVFSCSVMHEALHNHYT QKSLSLSPGK (SEQ ID NO: 57) |
| Human IgG4 (UniProt No. P01861) | ASTKGPSVFP LAPCSRSTSESTAALGCLVKDYFPEPVTVS WNSGALTSGVHTFPAVLQSSGLYSLSSVVTVPSSSLGTKT YTCNVDHKPSNTKVDKRVESKYGPPCPSCPAPEFLGGPSV FLFPPKPKDTLMISRTPEVTCVVVDVSQEDPEVQFNWYVD GVEVHNAKTKPREEQFNSTYRVVSVLTVLHQDWLNGKEYK CKVSNKGLPS SIEKTISKAKGQPREPQVYTLPPSQEEMTK NQVSLTCLVKGFYPSDIAVEWESNGQPENNYKTTPPVLDS DGSFFLYSRLTVDKSRWQEGNVFSCSVMHEALHNHYTQKS LSLSLGK (SEQ ID NO: 58) |
| Human IgG1 with silencing mutations (Fc region) | ASTKGPSVFPLAPSSKSTSGGTAALGCLVKDYFPEPVTVSWN SGALTSGVHTFPAVLQSSGLYSLSSVVTVPSSSLGTQTYICNV NHKPSNTKVDKKVEPKSCDKTHTCPPCPAPEAAGGPSVFLFP PKPKDTLMISRTPEVTCVVVDVSHEDPEVKFNWYVDGVEVH NAKTKPREEQYNSTYRVVSVLTVLHQDWLNGKEYKCKVSN KALPAPIEKTISKAKGQPREPQVYTLPPSREEMTKNQVSLTCL VKGFYPSDIAVEWESNGQPENNYKTTPPVLDSDGSFFLYSKL TVDKSRWQQGNVFSCSVMHEALHNHYTQKSLSLSPGK (SEQ ID NO: 59) |
| Human IgG4 with silencing mutations (Fc region) | ASTKGPSVFPLAPCSRSTSESTAALGCLVKDYFPEPVTVSWNS GALTSGVHTFPAVLQSSGLYSLSSVVTVPSSSLGTKTYTCNV DHKPSNTKVDKRVESKYGPPCPPCPAPEAAGGPSVFLFPPKP KDTLMISRTPEVTCVVVDVSQEDPEVQFNWYVDGVEVHNA KTKPREEQFNSTYRVVSVLTVLHQDWLNGKEYKCKVSNKGL PSSIEKTISKAKGQPREPQVYTLPPSQEEMTKNQVSLTCLVKG FYPSDIAVEWESNGQPENNYKTTPPVLDSDGSFFLYSRLTVD KSRWQEGNVFSCSVMHEALHNHYTQKSLSLSLGK (SEQ ID NO: 60) |
| Human IgG4 hinge region (wild type) | ESKYGPPCPSCPA (SEQ ID NO: 61) |
| Human IgG4 hinge region (S228P) | ESKYGPPCPPCPA (SEQ ID NO: 62) |
| Human IgG4 CH2 domain sequence (wild type) | APEFLGGPSVFLFPPKPKDTLMISRTPEVTCVVVDVSQEDPEV QFNWYVDGVEVHNAKTKPREEQFNSTYRVVSVLTVLHQDW LNGKEYKCKVSNKGLPSSIEKTISKAK (SEQ ID NO: 63) |
| Human IgG4 CH2 domain sequence (F234A, L235A) | APEAAGGPSVFLFPPKPKDTLMISRTPEVTCVVVDVSQEDPEV QFNWYVDGVEVHNAKTKPREEQFNSTYRVVSVLTVLHQDW LNGKEYKCKVSNKGLPSSIEKTISKAK (SEQ ID NO: 64) |
| Human IgG4 CH3 domain sequence (wild type) | GQPREPQVYTLPPSQEEMTKNQVSLTCLVKGFYPSDIAVEWE SNGQPENNYKTTPPVLDSDGSFFLYSRLTVDKSRWQEGNVFS CSVMHEALHNHYTQKSLSLSLGK (SEQ ID NO: 65) |
| Human IgG4 CH3 domain sequence (knob, T366W) | GQPREPQVYTLPPSQEEMTKNQVSLWCLVKGFYPSDIAVEW ESNGQPENNYKTTPPVLDSDGSFFLYSRLTVDKSRWQEGNVF SCSVMHEALHNHYTQKSLSLSLGK (SEQ ID NO: 66) |
| Human IgG4 CH3 domain sequence (hole, T366S, L368A, Y407V) | GQPREPQVYTLPPSQEEMTKNQVSLSCAVKGFYPSDIAVEWE SNGQPENNYKTTPPVLDSDGSFFLVSRLTVDKSRWQEGNVFS CSVMHEALHNHYTQKSLSLSLGK (SEQ ID NO: 67) |

TABLE S10

Example Human CD3 Epsilon and CD3 Delta Sequences

| | |
|---|---|
| Polypeptide Name | Amino Acid Sequence |
| Human CD3 epsilon | MQSGTHWRVLGLCLLSVGVWGQDGNEEMGGITQTPYKVSIS GTTVILTCPQYPGSEILWQHNDKNIGGDEDDKNIGSDEDHLSL KEFSELEQSGYYVCYPRGSKPEDANFYLYLRARVCENCMEM DVMSVATIVIVDICITGGLLLLVYYWSKNRKAKAKPVTRGAG AGGRQRGQNKERPPPVPNPDYEPIRKGQRDLYSGLNQRRI (SEQ ID NO: 69) |
| Human CD3 delta | MEHSTFLSGLVLATLLSQVSPFKIPIEELEDRVFVNCNTSITWV EGTVGTLLSDITRLDLGKRILDPRGIYRCNGTDIYKDKESTVQ VHYRMCQSCVELDPATVAGIIVTDVIATLLLALGVFCFAGHE TGRLSGAADTQALLRNDQVYQPLRDRDDAQYSHLGGNWAR NK (SEQ ID NO: 70) |

Some embodiments of the present disclosure relate to a polynucleotide encoding a single domain antibody that specifically binds to mesothelin as described herein.

Some embodiments of the present disclosure relate to a composition comprising one or more polynucleotide(s) encoding a mesothelin binding protein as described herein. In some embodiments, the mesothelin binding protein is an anti-mesothelin antibody or fragment thereof.

Some embodiments of the present disclosure relate to a recombinant expression vector comprising a single domain antibody that specifically binds to mesothelin as described herein, as well as a host cell comprising the recombinant expression vector.

Some embodiments of the present disclosure relate to one or more recombinant expression vector(s) comprising one or more polynucleotide(s) encoding a mesothelin binding protein as described herein, as well as a host cell comprising the one or more recombinant expression vector(s).

Some embodiments of the present disclosure relate to a synthetic immune receptor comprising a single domain antibody that specifically binds to mesothelin as described herein, as well as a cell comprising the synthetic immune receptor.

In some embodiments, the single domain antibody is connected to a transmembrane domain via a hinge region, and is further connected to a costimulatory domain, such as, e.g., a functional signaling domain obtained from OX40, CD27, CD28, CD5, ICAM-1, LFA-1 (CD11a/CD18), ICOS (CD278), or 4-1BB. In some embodiments, the synthetic immune receptor further comprises a sequence encoding a intracellular signaling domain, such as, e.g., 4-1BB and/or CD3 zeta.

Some embodiments of the present disclosure relate to producing a mesothelin binding protein (such as, e.g., an anti-mesothelin antibody or fragment thereof) described herein, comprising growing a cell described herein under conditions permissive for expression of the antibody, and isolating the mesothelin binding protein (such as, e.g., the anti-mesothelin antibody or fragment thereof) from the cell.

Some embodiments of the present disclosure relate to an antibody-drug conjugate comprising a single domain antibody that specifically binds to mesothelin, as described herein. In some embodiments, the drug in the antibody-drug conjugate is a chemotherapy agent. In some embodiments, the drug in the antibody-drug conjugate is a radionuclide. In some embodiments, the antibody-drug conjugate is for use in a diagnostic application, such as, e.g., the detection or monitoring of a disease associated with mesothelin expression, such as, e.g., a proliferative disease or cancer.

Some embodiments of the present disclosure relate to a pharmaceutical composition comprising a mesothelin binding protein, antibody-drug conjugate, or anti-mesothelin antibody or fragment thereof and a pharmaceutically acceptable excipient.

Non-limiting examples of pharmaceutically acceptable excipients include adjuvants, solid carriers, water, buffers, or other carriers used in the art to hold therapeutic components, or combinations thereof.

Pharmaceutical compositions of the present disclosure may be prepared for storage by mixing proteins having the desired degree of purity with optional pharmaceutically acceptable carriers, excipients or stabilizers (see, e.g. Remington’s Pharmaceutical Sciences 16th edition, Osol, A. Ed. (1980)), such as, e.g., in the form of lyophilized formulations or aqueous solutions. Acceptable carriers, excipients, or stabilizers are nontoxic to recipients at the dosages and concentrations employed, and include, but are not limited to, buffers such as phosphate, citrate, and other organic acids; antioxidants including ascorbic acid and methionine; preservatives (such as, e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition may comprise formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as, e.g., glycine, glutamine, asparagine, arginine or lysine); antimicrobials; antioxidants (such as, e.g., ascorbic acid, sodium sulfite or sodium hydrogensulfite); buffers (such as, e.g., borate, bicarbonate, Tris-HCl, citrates, phosphates or other organic acids); bulking agents (such as, e.g., mannitol or glycine); chelating agents (such as, e.g., ethylenediamine tetraacetic acid (EDTA)); complexing agents (such as, e.g., caffeine, polyvinylpyrrolidone, beta-cyclodextrin or hydroxypropyl-beta-cyclodextrin); fillers; monosaccharides; disaccharides; and other carbohydrates (such as, e.g., glucose, mannose or dextrins); proteins (such as, e.g., serum albumin, gelatin or immunoglobulins); coloring, flavoring and diluting agents; emulsifying agents; hydrophilic polymers (such as, e.g., polyvinylpyrrolidone); low molecular weight polypeptides; salt-forming counterions (such as, e.g., sodium); preservatives (such as, e.g., benzalkonium chloride, benzoic acid, salicylic acid, thimerosal, phenethyl alcohol, methylparaben, propylparaben, chlorhexidine, sorbic acid or hydrogen peroxide); solvents (such as, e.g., glycerin, propylene glycol or polyethylene glycol); sugar alcohols (such as, e.g., mannitol or sorbitol); suspending agents; surfactants or wetting agents (such as, e.g., pluronics, PEG, sorbitan esters, polysorbates such as, e.g., polysorbate 20, polysorbate 80, triton, tromethamine, lecithin, cholesterol, tyloxapal); stability enhancing agents (such as, e.g., sucrose or sorbitol); tonicity enhancing agents (such as, e.g., alkali metal halides, such as, e.g., sodium or potassium chloride, mannitol, sorbitol); delivery vehicles; diluents; excipients and/or pharmaceutical adjuvants. Methods and suitable materials for formulating molecules for therapeutic use are known in the pharmaceutical arts, and are described, for example, in REMINGTON’S PHARMACEUTICAL SCIENCES, 18th Edition, (A.R. Genrmo, ed.), 1990, Mack Publishing Company. Pharmaceutical compositions of the present disclosure include, but are not limited to, liquid, frozen, and lyophilized compositions.

Pharmaceutical compositions for parenteral administration are preferably sterile and substantially isotonic and manufactured under Good Manufacturing Practice (GMP) conditions. Pharmaceutical compositions can be provided in unit dosage form (such as, e.g., the dosage for a single administration). The formulation depends on the route of administration chosen. The mesothelin binding proteins (such as, e.g., anti-mesothelin antibodies and fragments thereof) and antibody-drug conjugates described herein can be administered by intravenous injection or infusion or subcutaneously. For injection administration, the mesothelin binding proteins (such as, e.g., anti-mesothelin antibodies and fragments thereof) and antibody-drug conjugates described herein can be formulated in aqueous solutions, preferably in physiologically-compatible buffers to reduce discomfort at the site of injection. The solution can contain carriers, excipients, or stabilizers as discussed above. Alternatively, mesothelin binding proteins (such as, e.g., anti-mesothelin antibodies and fragments thereof) and antibody-drug conjugates described herein can be in lyophilized form for reconstitution with a suitable vehicle, e.g., sterile pyrogen-free water, before use. The lyophilized material may be reconstituted in, e.g., bacteriostatic water for injection (BWFI), physiological saline, phosphate buffered saline (PBS), or the same formulation the protein had been in prior to lyophilization.

Antibody formulations are disclosed, for example, in U.S. Pat. No. 9,034,324. Similar formulations can be used for anti-mesothelin antibodies and fragments thereof described herein. Subcutaneous antibody formulations are described, for example, in US20160355591 and US20160166689.

Some embodiments of the present disclosure relate to a method of treating a disease associated with mesothelin expression in a subject in need thereof comprising administering to the subject a therapeutically effective dose of at least one mesothelin binding protein, antibody-drug conjugate, anti-mesothelin antibody, or antibody fragment as described herein.

In some embodiments, the administration results in slowing or inhibition of tumor growth or metastasis of a mesothelin-expressing cancer. Measurement of the reduction of the growth of tumor cells can be determined by multiple different methodologies that are well known in the art. Non-limiting examples include direct measurement of tumor dimension, measurement of excised tumor mass and comparison to control subjects, measurement via imaging techniques (such as, e.g., CT or MRI) that may or may not use isotopes or luminescent molecules (such as, e.g., luciferase) for enhanced analysis, and the like. In some embodiments, administration of the at least one mesothelin binding protein, antibody-drug conjugate, anti-mesothelin antibody, or antibody fragment results in a reduction of in vivo growth of tumor cells as compared to a control antigen binding agent by at least about 10%, 20%, 30%, 40%, 50%, 60%, 70%, 80%, 90%, or 100%, with an about 100% reduction in tumor growth indicating a complete response and disappearance of the tumor. In some embodiments, administration of the at least one mesothelin binding protein, antibody-drug conjugate, anti-mesothelin antibody, or antibody fragment results in a reduction of in vivo growth of tumor cells as compared to a control antigen binding agent by about 50-100%, about 75-100%, or about 90-100%. In some embodiments, administration of the at least one mesothelin binding protein, antibody-drug conjugate, anti-mesothelin antibody, or antibody fragment results in a reduction of in vivo growth of tumor cells as compared to a control antigen binding agent by about 50-60%, about 60-70%, about 70-80%, about 80-90%, or about 90-100%.

Effective doses for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject’s response to therapy. The amount of active ingredient that can be combined with the carrier materials to produce a single dosage form varies depending upon the host treated and the particular mode of administration.

In some embodiments, the mesothelin binding protein, antibody-drug conjugate, anti-mesothelin antibody, or antibody fragment is administered to the subject parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration.

In some embodiments, the mesothelin binding protein, antibody-drug conjugate, anti-mesothelin antibody, or antibody fragment is administered to the subject intravenously.

Parenteral or intravenous administration can be performed by injection (e.g. using a needle and a syringe) or by infusion (e.g. via a catheter and a pump system). It is envisaged that the administration according to the present disclosure is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

In some embodiments, the disease associated with mesothelin expression is chosen from proliferative diseases and cancer.

In some embodiments, the cancer is chosen from mesothelioma, pancreatic cancer, gastric cancer, ovarian cancer, lung cancer, and triple negative breast cancer.

Administration of a mesothelin binding protein, antibody-drug conjugate, anti-mesothelin antibody, or antibody fragment as described herein can also be accompanied by administration of other anti-cancer agents or therapeutic treatments (such as surgical resection of a tumor). Any suitable anti-cancer agent can be administered in combination with the mesothelin binding proteins, antibody-drug conjugates, anti-mesothelin antibodies, or antibody fragments disclosed herein. Example anti-cancer agents include, but are not limited to, chemotherapeutic agents, such as, e.g., mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (such as, e.g. anti-androgens) and anti-angiogenesis agents. Other anti-cancer treatments include radiation therapy and other antibodies that specifically target cancer cells.

In some embodiments, the mesothelin binding protein, antibody-drug conjugate, anti-mesothelin antibody, or antibody fragment is administered before, during, or after surgery.

Some embodiments of the present disclosure relate to a mesothelin binding protein, antibody-drug conjugate, anti-mesothelin antibody, or antibody fragment as described herein for use in the treatment of a disease associated with mesothelin expression. In some embodiments, the disease associated with mesothelin expression is chosen from proliferative diseases and cancer. In some embodiments, the cancer is chosen from mesothelioma, pancreatic cancer, gastric cancer, ovarian cancer, lung cancer, and triple negative breast cancer.

Some embodiments of the present disclosure relate to a use of a mesothelin binding protein, antibody-drug conjugate, anti-mesothelin antibody, or antibody fragment as described herein in the manufacture of a medicament for the treatment of a disease associated with mesothelin expression. In some embodiments, the disease associated with mesothelin expression is chosen from proliferative diseases and cancer. In some embodiments, the cancer is chosen from mesothelioma, pancreatic cancer, gastric cancer, ovarian cancer, lung cancer, and triple negative breast cancer.

Some embodiments of the present disclosure relate to a method of confirming the diagnosis of cancer in a subject by contacting a sample from the subject diagnosed with cancer with a single domain antibody or mesothelin binding protein described herein, and detecting binding of the single domain antibody or mesothelin binding protein to the sample. An increase in binding of the single domain antibody or mesothelin binding protein to the sample relative to binding of the single domain antibody or mesothelin binding protein to a control sample confirms the cancer diagnosis. In some embodiments, the method further includes contacting a detection antibody that specifically recognizes the single domain antibody or mesothelin binding protein with the sample, and detecting binding of the detection antibody.

Some embodiments of the present disclosure relate to a method of detecting a cancer associated with mesothelin expression in a subject. The method includes contacting a sample from the subject with a single domain antibody or mesothelin binding protein described herein, and detecting binding of the single domain antibody or mesothelin binding protein to the sample. An increase in binding of the single domain antibody or mesothelin binding protein to the sample relative to a control sample detects cancer in the subject. In some embodiments, the methods further comprise contacting a detection antibody that specifically recognizes the single domain antibody or mesothelin binding protein with the sample, and detecting binding of the detection antibody.

Some embodiments of the present disclosure relate to a kit comprising a mesothelin binding protein, antibody-drug conjugate, anti-mesothelin antibody, or antibody fragment as described herein or a pharmaceutical composition comprising the same and instructions for use. The kit can further contain a least one additional reagent, such as, e.g. a chemotherapeutic drug, etc. Kits typically include a label indicating the intended use of the contents of the kit. The term “label” as used herein includes any writing, or recorded material supplied on or with a kit, or which otherwise accompanies a kit.

In some embodiments, the kit is a diagnostic kit. In some embodiments, a kit is provided for detecting mesothelin in a biological sample, such as, e.g., a blood sample or tissue sample. For example, to confirm a cancer diagnosis in a subject, a biopsy can be performed to obtain a tissue sample for histological examination. Alternatively, a blood sample can be obtained to detect the presence of soluble mesothelin protein or fragment. Kits for detecting a polypeptide will typically comprise a single domain antibody or mesothelin binding protein according to the present disclosure. In some embodiments, the single domain antibody or mesothelin binding protein is labeled (such as, e.g., with a fluorescent, radioactive, or an enzymatic label).

In some embodiments, the kit may additionally comprise means for detecting a label (such as, e.g., enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

### Non-Limiting Example Embodiments

Without limitation, some example embodiments of this disclosure include:

1. A single domain antibody which specifically binds to mesothelin (MSLN), wherein the single domain antibody comprises a heavy chain variable (VH) region in which the full set of VH CDRs 1, 2, and 3 (combined) has at least 80% sequence identity to the CDRs 1, 2, and 3 of any one of SEQ ID NOs: 11-19.

2. The single domain antibody according to Embodiment 1, wherein the full set of VH CDRs 1, 2, and 3 (combined) has at least 85% sequence identity to the CDRs 1, 2, and 3 of any one of SEQ ID NOs: 11-19.

3. The single domain antibody according to Embodiment 1 or 2, wherein the full set of VH CDRs 1, 2, and 3 (combined) has at least 90% sequence identity to the CDRs 1, 2, and 3 of any one of SEQ ID NOs: 11-19.

4. The single domain antibody according to any one of Embodiments 1 to 3, wherein the full set of VH CDRs 1, 2, and 3 (combined) has at least 95% sequence identity to the CDRs 1, 2, and 3 of any one of SEQ ID NOs: 11-19.

5. A single domain antibody which specifically binds to mesothelin (MSLN), wherein the single domain antibody comprises a heavy chain variable (VH) region comprising:
(i) a VH complementarity determining region one (CDR1) comprising a sequence having at most two amino acid modifications relative to SEQ ID NO: 1 or SEQ ID NO: 9;
(ii) a VH CDR2 comprising a sequence having at most two amino acid modifications relative to SEQ ID NO: 2, SEQ ID NO: 7, or SEQ ID NO: 10; and
(iii) a VH CDR3 comprising a sequence having at most two amino acid modifications relative to SEQ ID NO: 3, SEQ ID NO: 4, SEQ ID NO: 5, SEQ ID NO: 6, or SEQ ID NO: 8.

6. The single domain antibody according to Embodiment 5, wherein each amino acid modification, if any, is a conservative amino acid substitution.

7. The single domain antibody according to Embodiment 5 or 6, wherein the VH CDR1 comprises a sequence having at most one amino acid modification relative to SEQ ID NO: 1 or SEQ ID NO: 9.

8. The single domain antibody according to any one of Embodiments 5 to 7, wherein the VH CDR2 comprises a sequence having at most one amino acid modification relative to SEQ ID NO: 2, SEQ ID NO: 7, or SEQ ID NO: 10.

9. The single domain antibody according to any one of Embodiments 5 to 8, wherein the VH CDR3 comprises a sequence having at most one amino acid modification relative to SEQ ID NO: 3, SEQ ID NO: 4, SEQ ID NO: 5, SEQ ID NO: 6, or SEQ ID NO: 8.

10. The single domain antibody according to any one of Embodiments 7 to 9, wherein the at most one amino acid modification is an amino acid substitution.

11. The single domain antibody according to any one of Embodiments 7 to 9, wherein the at most one amino acid modification is a conservative amino acid substitution.

12. The single domain antibody according to any one of Embodiments 7 to 9, wherein the at most one amino acid modification is an amino acid deletion.

13. The single domain antibody according to any one of Embodiments 7 to 9, wherein the at most one amino acid modification is an amino acid addition.

14. The single domain antibody according to any one of Embodiments 1 to 13, wherein the VH CDR1 comprises a sequence chosen from SEQ ID NO: 1 and SEQ ID NO: 9.

15. The single domain antibody according to any one of Embodiments 1 to 14, wherein the VH CDR2 comprises a sequence chosen from SEQ ID NO: 2, SEQ ID NO: 7, and SEQ ID NO: 10.

16. The single domain antibody according to any one of Embodiments 1 to 15, wherein the VH CDR3 comprises a sequence chosen from SEQ ID NO: 3, SEQ ID NO: 4, SEQ ID NO: 5, SEQ ID NO: 6, and SEQ ID NO: 8.

17. A single domain antibody which specifically binds to mesothelin (MSLN), wherein the single domain antibody comprises a heavy chain variable (VH) region comprising:
(i) a VH complementarity determining region one (CDR1) comprising the sequence G G S I S X₁ S Y Y (SEQ ID NO: 53), wherein X₁ is N or S;
(ii) a VH CDR2 comprising the sequence I Y X₂ S G X₃ X₄ (SEQ ID NO: 68), wherein X₂ is H or Y; X₃ is N or S; and X₄ is T or I; and
(iii) a VH CDR3 comprising the sequence X₅ X₆ Q X₇ G VG A T T TEE Y (SEQ ID NO: 54), wherein X₅ is T, V, or A; X₆ is S or T; and X₇ is D or N.

18. A single domain antibody which specifically binds to mesothelin (MSLN), wherein the single domain antibody comprises a heavy chain variable (VH) region comprising:
(i) a VH complementarity determining region one (CDR1) comprising a sequence chosen from SEQ ID NO: 1 and SEQ ID NO: 9;
(ii) a VH CDR2 comprising a sequence chosen from SEQ ID NO: 2, SEQ ID NO: 7, and SEQ ID NO: 10; and
(iii) a VH CDR3 comprising a sequence chosen from SEQ ID NO: 3, SEQ ID NO: 4, SEQ ID NO: 5, SEQ ID NO: 6, and SEQ ID NO: 8.

19. A single domain antibody which specifically binds to mesothelin (MSLN), wherein the single domain antibody comprises a heavy chain variable (VH) region comprising:
(a) a VH CDR1, a VH CDR2, and a VH CDR3 comprising the sequences of SEQ ID NOs: 1, 2, and 3, respectively;
(b) a VH CDR1, a VH CDR2, and a VH CDR3 comprising the sequences of SEQ ID NOs: 1, 2, and 4, respectively;
(c) a VH CDR1, a VH CDR2, and a VH CDR3 comprising the sequences of SEQ ID NOs: 1, 2, and 5, respectively;
(d) a VH CDR1, a VH CDR2, and a VH CDR3 comprising the sequences of SEQ ID NOs: 1, 2, and 6, respectively;
(e) a VH CDR1, a VH CDR2, and a VH CDR3 comprising the sequences of SEQ ID NOs: 1, 7, and 8, respectively;
(f) a VH CDR1, a VH CDR2, and a VH CDR3 comprising the sequences of SEQ ID NOs: 9, 2, and 6, respectively;
(g) a VH CDR1, a VH CDR2, and a VH CDR3 comprising the sequences of SEQ ID NOs: 9, 2, and 4, respectively; or
(h) a VH CDR1, a VH CDR2, and a VH CDR3 comprising the sequences of SEQ ID NOs: 1, 10, and 4, respectively.

20. A single domain antibody which specifically binds to mesothelin (MSLN), wherein the single domain antibody comprises a heavy chain variable (VH) region comprising the CDR1, CDR2, and CDR3 of any one of SEQ ID NOs: 11-19.

21. The single domain antibody according to any one of Embodiments 1 to 20, wherein the VH CDR1, VH CDR2, and VH CDR3 sequences are present in a human VH framework.

22. A single domain antibody which specifically binds to mesothelin (MSLN), wherein the single domain antibody comprises a heavy chain variable (VH) region having at least 80% sequence identity to any one of SEQ ID NOs: 11-19.

23. The single domain antibody according to any one of Embodiments 1 to 22, wherein the VH region has at least 85% sequence identity to any one of SEQ ID NOs: 11-19.

24. The single domain antibody according to any one of Embodiments 1 to 23, wherein the VH region has at least 90% sequence identity to any one of SEQ ID NOs: 11-19.

25. The single domain antibody according to any one of Embodiments 1 to 24, wherein the VH region has at least 95% sequence identity to any one of SEQ ID NOs: 11-19.

26. A single domain antibody which specifically binds to mesothelin (MSLN), wherein the single domain antibody comprises a heavy chain variable (VH) region chosen from SEQ ID NOs: 11-19.

27. The single domain antibody according to any one of Embodiments 1 to 26, wherein the single domain antibody specifically binds to human MSLN.

28. The single domain antibody according to any one of Embodiments 1 to 27, wherein the single domain antibody binds to human MSLN with a K_{D} of from about 10⁻⁹ M to about 10⁻⁶ M.

29. The single domain antibody according to any one of Embodiments 1 to 28, wherein the single domain antibody is an isolated single domain antibody.

30. An mesothelin binding protein comprising the single domain antibody according to any one of Embodiments 1 to 28.

31. The mesothelin binding protein according to Embodiment 30, wherein the mesothelin binding protein specifically binds to human MSLN.

32. The mesothelin binding protein according to Embodiment 30 or 31, wherein the mesothelin binding protein binds to human MSLN with a K_{D} of from about 10⁻⁹ M to about 10⁻⁶ M.

33. The mesothelin binding protein according to any one of Embodiments 30 to 32, wherein the mesothelin binding protein further specifically binds to CD3.

34. The mesothelin binding protein according to any one of Embodiments 30 to 33, wherein the mesothelin binding protein further specifically binds to human CD3.

35. The mesothelin binding protein according to any one of Embodiments 30 to 34, wherein the mesothelin binding protein further specifically binds to human CD3 epsilon.

36. The mesothelin binding protein according to any one of Embodiments 30 to 34, wherein the mesothelin binding protein binds to an epitope on CD3 comprising at least one residue selected from CD3 epsilon (SEQ ID NO: 69): K73 and S83; and CD3 delta (SEQ ID NO: 70) K82 and C93.

37. The mesothelin binding protein according to Embodiment 36, wherein the epitope on CD3 comprises the region of CD3 delta defined by K82, E83, S84, T85, V86, Q87, V88, H89, Y90, R91, M92, C93.

38. The mesothelin binding protein according to Embodiment 36 or 37, wherein the epitope on CD3 comprises the region of CD3 epsilon defined by K73, N74, 175, G76, S77, D78, E79, D80, H81, L82, S83.

39. The mesothelin binding protein according to any one of Embodiments 36 to 38, wherein the epitope comprises a conformational epitope with residues of both CD3 delta and CD3 epsilon.

40. The mesothelin binding protein according to any one of Embodiments 36 to 39, wherein the conformational epitope comprises each of residues CD3ε K73 and S83; CD3δ K82 and C93.

41. The mesothelin binding protein according to any one of Embodiments 30 to 40, wherein the mesothelin binding protein is a monoclonal antibody.

42. The mesothelin binding protein according to any one of Embodiments 30 to 41, wherein the mesothelin binding protein is an isolated monoclonal antibody.

43. An antibody-drug conjugate comprising the single domain antibody according to any one of Embodiments 1 to 28.

44. An anti-mesothelin antibody comprising the single domain antibody according to any one of Embodiments 1 to 28.

45. The anti-mesothelin antibody according to Embodiment 44, wherein the anti-mesothelin antibody binds to an effector cell.

46. The anti-mesothelin antibody according to Embodiment 44 or 45, wherein the anti–mesothelin antibody is multi-specific.

47. The anti-mesothelin antibody according to any one of Embodiments 44 to 46, wherein the anti-mesothelin antibody further specifically binds to a tumor-specific antigen other than MSLN.

48. The anti-mesothelin antibody according to any one of Embodiments 44 to 47, wherein the anti-mesothelin antibody is bispecific.

49. The anti-mesothelin antibody according to any one of Embodiments 44 to 48, wherein the anti-mesothelin antibody further specifically binds to CD3.

50. The anti-mesothelin antibody according to any one of Embodiments 44 to 49, wherein the anti-mesothelin antibody further specifically binds to human CD3.

51. The anti-mesothelin antibody according to any one of Embodiments 44 to 50, wherein the anti-mesothelin antibody further specifically binds to human CD3 epsilon.

52. The anti-mesothelin antibody according to any one of Embodiments 44 to 50, wherein the anti-mesothelin antibody binds to an epitope on CD3 comprising at least one residue selected from CD3 epsilon (SEQ ID NO: 69): K73 and S83; and CD3 delta (SEQ ID NO: 70) K82 and C93.

53. The anti-mesothelin antibody according to Embodiment 52, wherein the epitope on CD3 comprises the region of CD3 delta defined by K82, E83, S84, T85, V86, Q87, V88, H89, Y90, R91, M92, C93.

54. The anti-mesothelin antibody according to Embodiment 52 or 53, wherein the epitope on CD3 comprises the region of CD3 epsilon defined by K73, N74, 175, G76, S77, D78, E79, D80, H81, L82, S83.

55. The anti-mesothelin antibody according to any one of Embodiments 52 to 54, wherein the epitope comprises a conformational epitope with residues of both CD3 delta and CD3 epsilon.

56. The anti-mesothelin antibody according to Embodiment 55, wherein the conformational epitope comprises each of residues CD3ε K73 and S83; CD3δ K82 and C93.

57. The anti-mesothelin antibody according to any one of Embodiments 30 to 56, wherein the anti-mesothelin antibody further comprises a CD3-binding VH region.

58. The anti-mesothelin antibody according to any one of Embodiments 30 to 57, wherein the anti-mesothelin antibody is an IgG4 antibody.

59. The anti-mesothelin antibody according to any one of Embodiments 30 to 57, wherein the anti-mesothelin antibody is an IgG1 antibody.

60. The anti-mesothelin antibody according to any one of Embodiments 30 to 58, wherein the anti-mesothelin antibody further comprises a CD3-binding VH region that is paired with a light chain variable (LV) region.

61. The anti-mesothelin antibody according to Embodiment 57 or 60, wherein the CD3-binding VH region comprises:
(i) a VH complementarity determining region one (CDR1) comprising a sequence having at most two amino acid modifications relative to any one of SEQ ID NOs: 20-25;
(ii) a VH CDR2 comprising a sequence having at most two amino acid modifications relative to SEQ ID NO: 26; and
(iii) a VH CDR3 comprising a sequence having at most two amino acid modifications relative to any one of SEQ ID NOs: 27-30.

62. The anti-mesothelin antibody according to Embodiment 61, wherein the CD3-binding VH CDR1 comprises a sequence having at most one amino acid modification relative to any one of SEQ ID NO: 20-25.

63. The anti-mesothelin antibody according to Embodiment 61 or 62, wherein the CD3-binding VH CDR2 comprises a sequence having at most one amino acid modification relative to SEQ ID NO: 26.

64. The anti-mesothelin antibody according to any one of Embodiments 61 to 63, wherein the CD3-binding VH CDR3 comprises a sequence having at most one amino acid modification relative to any one of SEQ ID NOs: 27-30.

65. The anti-mesothelin antibody according to any one of Embodiments 62 to 64, wherein the at most one amino acid modification is an amino acid substitution.

66. The anti-mesothelin antibody according to any one of Embodiments 62 to 65, wherein the at most one amino acid modification is a conservative amino acid substitution.

67. The anti-mesothelin antibody according to any one of Embodiments 62 to 64, wherein the at most one amino acid modification is an amino acid deletion.

68. The anti-mesothelin antibody according to any one of Embodiments 62 to 64, wherein the at most one amino acid modification is an amino acid addition.

69. The anti-mesothelin antibody according to any one of Embodiments 62 to 68, wherein the CD3-binding VH CDR1 comprises a sequence chosen from SEQ ID NOs: 20-25.

70. The anti-mesothelin antibody according to any one of Embodiments 62 to 69, wherein the CD3-binding VH CDR2 comprises the sequence of SEQ ID NO: 26.

71. The anti-mesothelin antibody according to any one of Embodiments 62 to 70, wherein the CD3-binding VH CDR3 comprises a sequence chosen from SEQ ID NOs: 27-30.

72. The anti-mesothelin antibody according to any one of Embodiments 57 or 60 to 71, wherein the full set of VH CDRs 1, 2, and 3 (combined) in the CD3-binding VH region has at least 80% sequence identity to the CDRs 1, 2, and 3 of any one of SEQ ID NOs: 31-48.

73. The anti-mesothelin antibody according to any one of Embodiments 57 or 60 to 72, wherein the full set of VH CDRs 1, 2, and 3 (combined) in the CD3-binding VH region has at least 85% sequence identity to the CDRs 1, 2, and 3 of any one of SEQ ID NOs: 31-48.

74. The anti-mesothelin antibody according to any one of Embodiments 57 or 60 to 73, wherein the full set of VH CDRs 1, 2, and 3 (combined) in the CD3-binding VH region has at least 90% sequence identity to the CDRs 1, 2, and 3 of any one of SEQ ID NOs: 31-48.

75. The anti-mesothelin antibody according to any one of Embodiments 57 or 60 to 74, wherein the full set of VH CDRs 1, 2, and 3 (combined) in the CD3-binding VH region has at least 95% sequence identity to the CDRs 1, 2, and 3 of any one of SEQ ID NOs: 31-48.

76. The anti-mesothelin antibody according to Embodiment 57 or 60, wherein the CD3-binding VH region comprises:
(i) a VH complementarity determining region one (CDR1) comprising the sequence GF TF X₈ X₉ Y A (SEQ ID NO: 55), wherein X₈ is D, A, or H and X₉ is D or N;
(ii) a VH CDR2 comprising the sequence ISWNSGSI (SEQ ID NO: 26); and
(iii) a VH CDR3 comprising the sequence A K D S R G Y G X₁₀ Y X₁₁ X₁₂ G G A Y (SEQ ID NO: 56), wherein X₁₀ is D or S; X₁₁ is R or S; and X₁₂ is L or R.

77. The anti-mesothelin antibody according to Embodiment 57 or 60, wherein the CD3-binding VH region comprises the CDR1, CDR2, and CDR3 of any one of SEQ ID NOs: 31-48.

78. The anti-mesothelin antibody according to any one of Embodiments 57 to 77, wherein the VH CDR1, VH CDR2, and VH CDR3 sequences in the CD3-binding VH region are present in a human VH framework.

79. The anti-mesothelin antibody according to any one of Embodiments 57 to 78, wherein the CD3-binding VH region has at least 80% sequence identity to any one of SEQ ID NOs: 31-48.

80. The anti-mesothelin antibody according to any one of Embodiments 57 to 79, wherein the CD3-binding VH region has at least 85% sequence identity to any one of SEQ ID NOs: 31-48.

81. The anti-mesothelin antibody according to any one of Embodiments 57 to 80, wherein the CD3-binding VH region has at least 90% sequence identity to any one of SEQ ID NOs: 31-48.

82. The anti-mesothelin antibody according to any one of Embodiments 57 to 81, wherein the CD3-binding VH region has at least 95% sequence identity to any one of SEQ ID NOs: 31-48.

83. The anti-mesothelin antibody according to any one of Embodiments 57 to 82, wherein the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

84. The anti-mesothelin antibody according to Embodiment 83, wherein the CD3-binding VH region comprises a VH CDR1, a VH CDR2, and a VH CDR3 comprising the sequences of SEQ ID NOs: 20, 26, and 27.

85. The anti-mesothelin antibody according to any one of Embodiments 60 to 84, wherein the light chain variable region comprises the CDR1, CDR2, and CDR3 of SEQ ID NO: 52.

86. The anti-mesothelin antibody according to any one of Embodiments 60 to 85, wherein the light chain variable region comprises a VL CDR1, a VL CDR2, and a VL CDR3 comprising the sequence of SEQ ID NOs: 49, 50, and 51, respectively.

87. The anti-mesothelin antibody according to any one of Embodiments 60 to 86, wherein the VL CDR1, VL CDR2, and VL CDR3 sequences are present in a human VH framework.

88. The anti-mesothelin antibody according to any one of Embodiments 60 to 87, wherein the light chain variable region has at least 80% sequence identity to SEQ ID NO: 52.

89. The anti-mesothelin antibody according to any one of Embodiments 60 to 88, wherein the light chain variable region has at least 85% sequence identity to SEQ ID NO: 52.

90. The anti-mesothelin antibody according to any one of Embodiments 60 to 89, wherein the light chain variable region has at least 90% sequence identity to SEQ ID NO: 52.

91. The anti-mesothelin antibody according to any one of Embodiments 60 to 90, wherein the light chain variable region has at least 95% sequence identity to SEQ ID NO: 52.

92. The anti-mesothelin antibody according to any one of Embodiments 44 to 91, wherein the anti-mesothelin antibody further comprises a Fc region.

93. The anti-mesothelin antibody according to any one of Embodiments 44 to 92, wherein the anti-mesothelin antibody further comprises a variant Fc region.

94. The anti-mesothelin antibody according to Embodiment 93, wherein the variant Fc region comprises heterodimerizing alterations.

95. The anti-mesothelin antibody according to any one of Embodiments 44 to 94, wherein the Fc region is a silenced Fc region.

96. The anti-mesothelin antibody according to any one of Embodiments 44 to 95, wherein the anti-mesothelin antibody specifically binds to human MSLN.

97. The anti-mesothelin antibody according to any one of Embodiments 44 to 96, wherein the anti-mesothelin antibody binds to human MSLN with a K_{D} of from about 10⁻⁹ M to about 10⁻⁶ M.

98. The anti-mesothelin antibody according to any one of Embodiments 44 to 97, wherein the anti-mesothelin antibody is an isolated antibody.

99. An antibody fragment that specifically binds to mesothelin, wherein the antibody fragment comprises a fragment of the anti-mesothelin antibody according to any one of Embodiments 44 to 98.

100. The antibody fragment according to Embodiment 99, wherein the antibody fragment specifically binds to human MSLN.

101. The antibody fragment according to Embodiment 99 or 100, wherein the antibody fragment is an isolated antibody fragment.

102. A polynucleotide encoding the single domain antibody according to any one of Embodiments 1 to 29.

103. A composition comprising one or more polynucleotide(s) encoding the mesothelin binding protein according to any one of Embodiments 30 to 42.

104. A composition comprising one or more polynucleotide(s) encoding the anti-mesothelin antibody according to any one of Embodiments 44 to 98.

105. A recombinant expression vector comprising the polynucleotide or composition according to any one of Embodiments 102 to 104.

106. A host cell comprising the recombinant expression vector according to Embodiment 105.

107. A synthetic immune receptor comprising the single domain antibody according to any one of Embodiments 1 to 28.

108. A cell comprising the synthetic immune receptor according to Embodiment 107.

109. A method of treating a disease associated with mesothelin expression in a subject in need thereof comprising administering to the subject at least one mesothelin binding protein, antibody-drug conjugate, anti-mesothelin antibody, or antibody fragment according to any one of Embodiments 30 to 101.

110. A method of treating a disease associated with mesothelin expression in a subject in need thereof comprising administering to the subject a therapeutically effective dose of at least one mesothelin binding protein, antibody-drug conjugate, anti-mesothelin antibody, or antibody fragment according to any one of Embodiments 30 to 101.

111. The method according to Embodiment 110, wherein the disease associated with mesothelin expression is chosen from proliferative diseases and cancer.

112. The method according to Embodiment 111, wherein the cancer is chosen from mesothelioma, pancreatic cancer, gastric cancer, ovarian cancer, lung cancer, and triple negative breast cancer.

113. A mesothelin binding protein, antibody-drug conjugate, anti-mesothelin antibody, or antibody fragment according to any one of Embodiments 30 to 101 for use in the treatment of a disease associated with mesothelin expression.

114. The mesothelin binding protein, antibody-drug conjugate, anti-mesothelin antibody, or antibody fragment for use according to Embodiment 113, wherein the disease associated with mesothelin expression is chosen from proliferative diseases and cancer.

115. The mesothelin binding protein, antibody-drug conjugate, anti-mesothelin antibody, or antibody fragment for use according to Embodiment 114, wherein the cancer is chosen from mesothelioma, pancreatic cancer, gastric cancer, ovarian cancer, lung cancer, and triple negative breast cancer.

116. Use of a mesothelin binding protein, antibody-drug conjugate, anti-mesothelin antibody, or antibody fragment according to any one of Embodiments 30 to 101 in the manufacture of a medicament for the treatment of a disease associated with mesothelin expression.

117. The use according to Embodiment 116, wherein the disease associated with mesothelin expression is chosen from proliferative diseases and cancer.

118. The use according to Embodiment 117, wherein the cancer is chosen from mesothelioma, pancreatic cancer, gastric cancer, ovarian cancer, lung cancer, and triple negative breast cancer.

119. A pharmaceutical composition comprising at least one mesothelin binding protein, antibody-drug conjugate, anti-mesothelin antibody, or antibody fragment according to any one of Embodiments 30 to 101 and a pharmaceutically acceptable excipient.

### EXAMPLES

In order that the present disclosure may be more fully understood, the following examples are set forth. It should be understood that these examples are for illustrative purposes only and are not to be construed as limiting this disclosure in any manner.

### Example 1. Binding to MSLN-Expressing Cell Line and Off-Target Cell Line

Cell binding for example anti-MSLN single domain antibodies of the disclosure was assessed using CHO cells expressing human MSLN (CHO_huMSLN) and CHO cells that do not express MSLN protein (CHO-OFFtgt). Pelleted CHO cells were resuspended in flow buffer (1X phosphate buffered saline (PBS), 1% bovine serum albumin (BSA), and 0.1% NaN₃) at 1.25 × 10⁶ cells/mL. Antibody containing supernatants were diluted 1:5 in flow buffer, and 40 µL cells plus 10 µL diluted antibody supernatant were incubated for 30 minutes at 4° C. Cells were washed with flow buffer and resuspended in 50 µL phycoerythrin (PE)-conjugated secondary antibody (Southern Biotech #2042-09) diluted in flow buffer to 1.25 µg/mL and incubated for 20 minutes at 4° C. After two wash steps, cells were resuspended in flow buffer and analyzed using a Guava easyCyte 8HT system. Table E1 summarizes the target binding activity of the anti-MSLN single domain antibodies as fold over background MFI signal.

TABLE E1

Binding to MSLN-Expressing Cell Line and Off-Target Cell Line

| Clone ID # | CHO_huMSLN | CHO_OFFtgt |
|---|---|---|
| 394556 | 540.4 | 1.1 |
| 394541 | 422.5 | 1.0 |
| 394582 | 367.3 | 1.0 |
| 392026 | 347.0 | 1.0 |
| 394573 | 193.7 | 1.1 |
| 394607 | 140.6 | 1.1 |
| 394606 | 87.3 | 1.0 |
| 394610 | 78.0 | 1.0 |
| 394567 | 16.9 | 1.1 |

### Example 2. Binding to MSLN-Expressing Cell Lines and Off-Target Cell Line

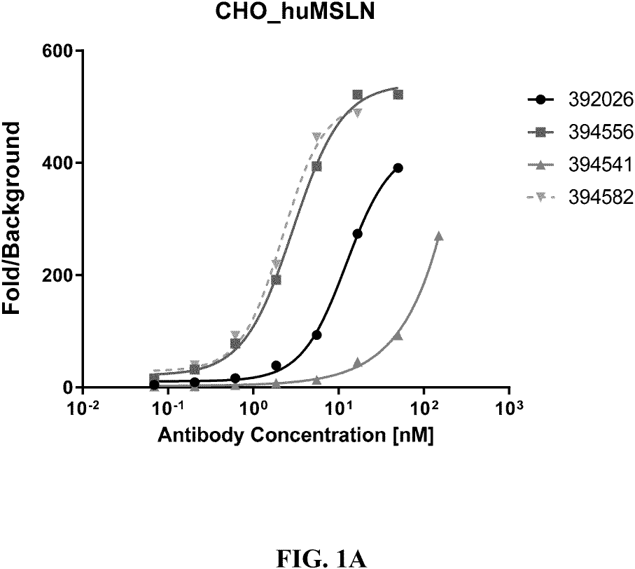

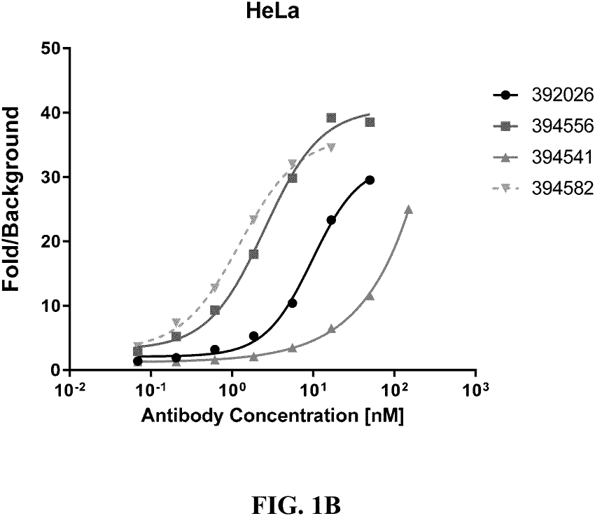

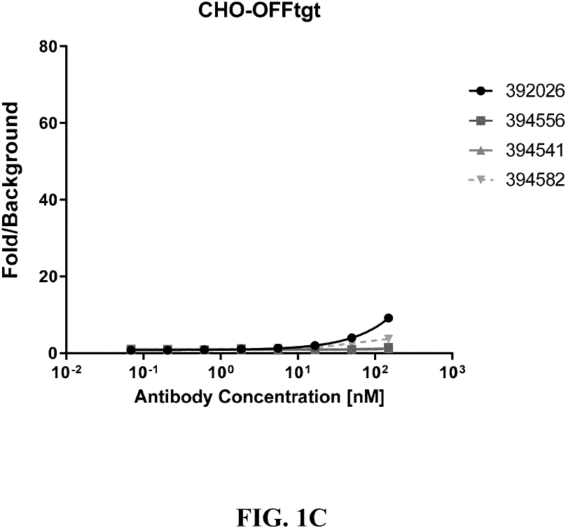

Cell-binding dose curves for example anti-MSLN single domain antibodies described herein were performed using CHO cells expressing human MSLN (FIG. 1A), HeLa cells (FIG. 1B), and CHO-OFFtgt cells (FIG. 1C). The single domain antibodies were tested at a starting concentration of 150 nM, followed by 3-fold serial dilutions for an 8-point dose curve. All washes and dilutions of cells, antibodies, and reagents were performed using flow buffer (1X phosphate buffered saline (PBS), 1% bovine serum albumin (BSA), and 0.1% NaN₃). Cells were pelleted and resuspended at 1 × 10⁶ cells/mL in flow buffer. Then, 50 µL cells were combined with 50 µL of test antibody and incubated 30 minutes at 4° C., followed by two wash steps with flow buffer. Cells were then resuspended in 50 µL PE-conjugated secondary diluted to 1.25 µg/mL (Southern Biotech #2042-09) and incubated for 20 minutes at 4° C. After two wash steps, cells were resuspended in flow buffer and analyzed using a BD Celesta system. PE mean fluorescence intensity was plotted as a fold over background (cells incubated with secondary detection antibody only).

### Example 3. Cell Binding EC50 Values on MSLN-Expressing Cell Lines

To determine cell binding EC50 values for example anti-MSLN single domain antibodies of the present disclosure, cell binding dose curves were performed on HeLa cells and CHO cells expressing human MSLN. The single domain antibodies were tested at a starting dose of 150 nM, followed by 3-fold serial dilutions for an 8-point dose curve as described in Example 2. The transformed data were plotted as an xy-graph using the non-linear regression curve fit (available in GraphPad Prism 8.4.3) to obtain the EC50 values (nM), which are summarized in Table E2.

TABLE E2

Cell Binding EC50 Values on MSLN-Expressing Cell Lines

| Clone ID # | CHO_huMSLN EC50 | HeLa EC50 |
|---|---|---|
| 394556 | 2.9 | 2.5 |
| 394541 | ND | ND |
| 394582 | 2.2 | 1.3 |
| 392026 | 12.4 | 9.8 |

### Example 4. Binding Affinity and Epitope Binning

Table E3 summarizes affinity and epitope bin information for example anti-MSLN single domain antibodies of the present disclosure. The affinity of each single domain antibody to recombinant human MSLN (R&D Systems, #3265-MS) was measured by Bio-layer interferometry (BLI) using an Octet HTX. AHC (Anti-hIgG Fc Capture) sensors were used to immobilize test antibody at 5 µg/mL. After baseline readings, sensors were dipped into antigen solutions (starting at 500 nM followed by a 7-point, 2-fold dilution series). Association and dissociation were measured for 180 and 240 seconds respectively. Data were analyzed with Octet Data Analysis v11.0 HT (ForteBio) using a standard 1:1 binding model. Epitope bins for the single domain antibodies were determined by an in-tandem competition BLI binding experiment using an Octet HTX. The antigen (5 µg/mL) was loaded on the sensor Ni-NTA sensor followed by a baseline in kinetic buffer. The antigen coated sensor was then dipped into a saturating concentration Antibody 1. A second baseline was now set for 300 sec. Then the antigen-antibody 1 complex was dipped into Antibody 2 solution for 180 sec and then allowed to dissociate.

TABLE E3

Binding Affinity and Epitope Binning

| Clone ID # | KD (M) | Epitope Bin |
|---|---|---|
| 394556 | 9.04E-08 | 1 |
| 394541 | 3.80E-06 | 1 |
| 394582 | 2.20E-07 | 1 |
| 392026 | no binding | 1 |

### Example 5. CAR-T Mediated T-Cell Activation by Human Tumor Cell Lines

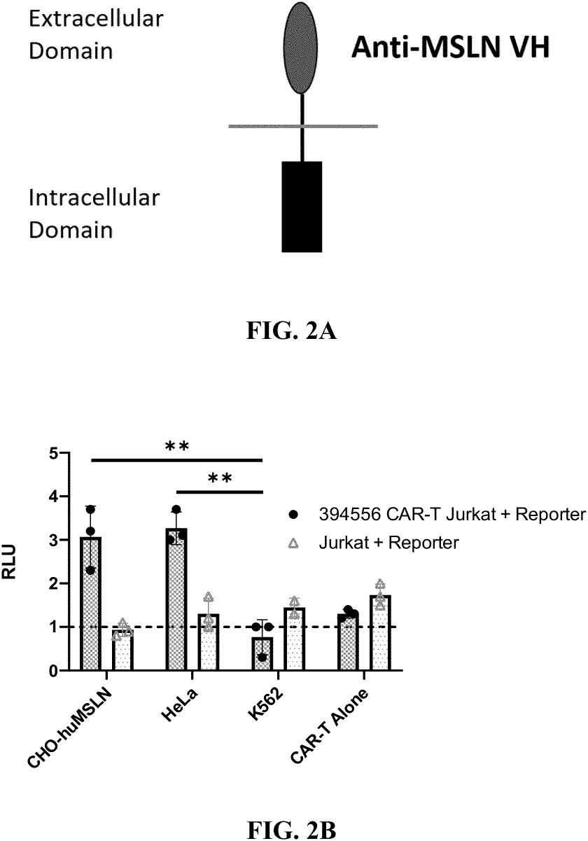

CAR-T cell activity was measured by transfecting Jurkat T lymphocyte cells with an anti-MSLN CAR and a 6x NFAT TK nano luciferase reporter according to the manufacturer’s protocol (Lonza 4D-Nucleofector X; SE Cell Line 4D-Nucleofector X Kit L, # V4XC-1012; program CL-120). FIG. 2A is a schematic illustration of an example CAR-T structure comprising an anti-MSLN extracellular binding domain comprising an antibody sequence of the present disclosure. Transfected Jurkat cells were co-cultured in RPMI-1640 (Fisher Scientific, Waltham, Massachusetts, USA) with 10% FBS (ThermoFisher) for 24 hours in a humidified, 37° C., 8% CO₂ incubator with MSLN+ CHO cells stably transfected to express human MSLN, HeLa, or MSLN-negative K562 cells. Luciferase activity was measured on a SpectraMax i3x multimode microplate reader (Molecular Devices) using the Promega Nano-Glo Luciferase Assay System according to the manufacturer’s protocol (Catalog # N1110) and data were normalized to co-culture containing the CAR transfected Jurkat and MSLN negative K562 cell lines. Statistical significance was determined using an unpaired, two-tailed t-test. The results are provided in FIG. 2B.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A single domain antibody which specifically binds to mesothelin (MSLN), wherein the single domain antibody comprises a heavy chain variable (VH) region in which the full set of VH CDRs 1, 2, and 3 (combined) has at least 95% sequence identity to the CDRs 1, 2, and 3 of any one of SEQ ID NOs: 11-19.

2. A single domain antibody which specifically binds to mesothelin (MSLN), wherein the single domain antibody comprises a heavy chain variable (VH) region comprising:
(i) a VH complementarity determining region one (CDR1) comprising a sequence having at most two amino acid modifications relative to SEQ ID NO: 1 or SEQ ID NO: 9;
(ii) a VH CDR2 comprising a sequence having at most two amino acid modifications relative to SEQ ID NO: 2, SEQ ID NO: 7, or SEQ ID NO: 10; and
(iii) a VH CDR3 comprising a sequence having at most two amino acid modifications relative to SEQ ID NO: 3, SEQ ID NO: 4, SEQ ID NO: 5, SEQ ID NO: 6, or SEQ ID NO: 8.

3. A single domain antibody which specifically binds to mesothelin (MSLN), wherein the single domain antibody comprises a heavy chain variable (VH) region comprising:
(i) a VH complementarity determining region one (CDR1) comprising the sequence G G S I S X₁ S Y Y (SEQ ID NO: 53),
wherein X₁ is N or S;
(ii) a VH CDR2 comprising the sequence I Y X₂ S G X₃ X₄ (SEQ ID NO: 68),
wherein X₂ is H or Y; X₃ is N or S; and X₄ is T or I; and
(iii) a VH CDR3 comprising the sequence X₅ X₆ Q X₇ G VG A T T TEE Y (SEQ ID NO: 54),
wherein X₅ is T, V, or A; X₆ is S or T; and X₇ is D or N.

4. A single domain antibody which specifically binds to mesothelin (MSLN), wherein the single domain antibody comprises a heavy chain variable (VH) region comprising:
(i) a VH complementarity determining region one (CDR1) comprising a sequence chosen from SEQ ID NO: 1 and SEQ ID NO: 9;
(ii) a VH CDR2 comprising a sequence chosen from SEQ ID NO: 2, SEQ ID NO: 7, and SEQ ID NO: 10; and
(iii) a VH CDR3 comprising a sequence chosen from SEQ ID NO: 3, SEQ ID NO: 4, SEQ ID NO: 5, SEQ ID NO: 6, and SEQ ID NO: 8.

5. A single domain antibody which specifically binds to mesothelin (MSLN), wherein the single domain antibody comprises a heavy chain variable (VH) region comprising:
(a) a VH CDR1, a VH CDR2, and a VH CDR3 comprising the sequences of SEQ ID NOs: 1, 2, and 3, respectively;
(b) a VH CDR1, a VH CDR2, and a VH CDR3 comprising the sequences of SEQ ID NOs: 1, 2, and 4, respectively;
(c) a VH CDR1, a VH CDR2, and a VH CDR3 comprising the sequences of SEQ ID NOs: 1, 2, and 5, respectively;
(d) a VH CDR1, a VH CDR2, and a VH CDR3 comprising the sequences of SEQ ID NOs: 1, 2, and 6, respectively;
(e) a VH CDR1, a VH CDR2, and a VH CDR3 comprising the sequences of SEQ ID NOs: 1, 7, and 8, respectively;
(f) a VH CDR1, a VH CDR2, and a VH CDR3 comprising the sequences of SEQ ID NOs: 9, 2, and 6, respectively;
(g) a VH CDR1, a VH CDR2, and a VH CDR3 comprising the sequences of SEQ ID NOs: 9, 2, and 4, respectively; or
(h) a VH CDR1, a VH CDR2, and a VH CDR3 comprising the sequences of SEQ ID NOs: 1, 10, and 4, respectively.

6. A single domain antibody which specifically binds to mesothelin (MSLN), wherein the single domain antibody comprises a heavy chain variable (VH) region comprising the CDR1, CDR2, and CDR3 of any one of SEQ ID NOs: 11-19.

7. The single domain antibody according to any one of claims 1 to 6, wherein the VH CDR1, VH CDR2, and VH CDR3 sequences are present in a human VH framework.

8. A single domain antibody which specifically binds to mesothelin (MSLN), wherein the single domain antibody comprises a heavy chain variable (VH) region having at least 95% sequence identity to any one of SEQ ID NOs: 11-19.

9. A single domain antibody which specifically binds to mesothelin (MSLN), wherein the single domain antibody comprises a heavy chain variable (VH) region chosen from SEQ ID NOs: 11-19.

10. The single domain antibody according to any one of claims 1 to 9, wherein the single domain antibody specifically binds to human MSLN.

11. The single domain antibody according to any one of claims 1 to 10, wherein the single domain antibody binds to human MSLN with a K_{D} of from about 10⁻⁹ M to about 10⁻⁶ M.

12. The single domain antibody according to any one of claims 1 to 11, wherein the single domain antibody is an isolated single domain antibody.

13. An mesothelin binding protein comprising the single domain antibody according to any one of claims 1 to 11.

14. The mesothelin binding protein according to claim 13, wherein the mesothelin binding protein specifically binds to human MSLN.

15. The mesothelin binding protein according to claim 13 or 14, wherein the mesothelin binding protein further specifically binds to CD3.

16. The mesothelin binding protein according to any one of claims 13 to 15, wherein the mesothelin binding protein further specifically binds to human CD3.

17. The mesothelin binding protein according to any one of claims 13 to 16, wherein the mesothelin binding protein is an isolated monoclonal antibody.

18. An antibody-drug conjugate comprising the single domain antibody according to any one of claims 1 to 11.

19. An anti-mesothelin antibody comprising the single domain antibody according to any one of claims 1 to 11.

20. The anti-mesothelin antibody according to claim 19, wherein the anti-mesothelin antibody is multi-specific.

21. The anti-mesothelin antibody according to claim 19 or 20, wherein the anti-mesothelin antibody further specifically binds to a tumor-specific antigen other than MSLN.

22. The anti-mesothelin antibody according to any one of claims 19 to 21, wherein the anti-mesothelin antibody is bispecific.

23. The anti-mesothelin antibody according to any one of claims 19 to 22, wherein the anti-mesothelin antibody further specifically binds to CD3.

24. The anti-mesothelin antibody according to any one of claims 19 to 23, wherein the anti-mesothelin antibody further specifically binds to human CD3.

25. The anti-mesothelin antibody according to any one of claims 19 to 24, wherein the anti-mesothelin antibody further comprises a CD3-binding VH region.

26. The anti-mesothelin antibody according to any one of claims 19 to 25, wherein the anti-mesothelin antibody is of the IgG1 or IgG4 subtype.

27. The anti-mesothelin antibody according to any one of claims 19 to 26, wherein the anti-mesothelin antibody further comprises a CD3-binding VH region that is paired with a light chain variable (LV) region.

28. The anti-mesothelin antibody according to claim 25 or 27, wherein the CD3-binding VH region comprises:
(i) a VH complementarity determining region one (CDR1) comprising a sequence having at most two amino acid modifications relative to any one of SEQ ID NOs: 20-25;
(ii) a VH CDR2 comprising a sequence having at most two amino acid modifications relative to SEQ ID NO: 26; and
(iii) a VH CDR3 comprising a sequence having at most two amino acid modifications relative to any one of SEQ ID NOs: 27-30.

29. The anti-mesothelin antibody according to claim 28, wherein the CD3-binding VH CDR1 comprises a sequence chosen from SEQ ID NOs: 20-25.

30. The anti-mesothelin antibody according to claim 28 or 29, wherein the CD3-binding VH CDR2 comprises the sequence of SEQ ID NO: 26.

31. The anti-mesothelin antibody according to any one of claims 28 to 30, wherein the CD3-binding VH CDR3 comprises a sequence chosen from SEQ ID NOs: 27-30.

32. The anti-mesothelin antibody according to any one of claims 25 or 27 to 31, wherein the full set of VH CDRs 1, 2, and 3 (combined) in the CD3-binding VH region has at least 95% sequence identity to the CDRs 1, 2, and 3 of any one of SEQ ID NOs: 31-48.

33. The anti-mesothelin antibody according to claim 25 or 27, wherein the CD3-binding VH region comprises:
(i) a VH complementarity determining region one (CDR1) comprising the sequence G F T F X₈ X₉ Y A (SEQ ID NO: 55),
wherein Xs is D, A, or H and X₉ is D or N;
(ii) a VH CDR2 comprising the sequence ISWNSGSI (SEQ ID NO: 26); and
(iii) a VH CDR3 comprising the sequence A K D S R G Y G X₁₀ Y X₁₁ X₁₂ G G A Y (SEQ ID NO: 56),
wherein X₁₀ is D or S; X₁₁ is R or S; and X₁₂ is L or R.

34. The anti-mesothelin antibody according to claim 25 or 27, wherein the CD3-binding VH region comprises the CDR1, CDR2, and CDR3 of any one of SEQ ID NOs: 31-48.

35. The anti-mesothelin antibody according to any one of claims 25 to 34, wherein the VH CDR1, VH CDR2, and VH CDR3 sequences in the CD3-binding VH region are present in a human VH framework.

36. The anti-mesothelin antibody according to any one of claims 25 to 35, wherein the CD3-binding VH region has at least 95% sequence identity to any one of SEQ ID NOs: 31-48.

37. The anti-mesothelin antibody according to any one of claims 25 to 36, wherein the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

38. The anti-mesothelin antibody according to claim 37, wherein the CD3-binding VH region comprises a VH CDR1, a VH CDR2, and a VH CDR3 comprising the sequences of SEQ ID NOs: 20, 26, and 27.

39. The anti-mesothelin antibody according to any one of claims 27 to 38, wherein the light chain variable region comprises the CDR1, CDR2, and CDR3 of SEQ ID NO: 52.

40. The anti-mesothelin antibody according to any one of claims 27 to 39, wherein the light chain variable region comprises a VL CDR1, a VL CDR2, and a VL CDR3 comprising the sequence of SEQ ID NOs: 49, 50, and 51, respectively.

41. The anti-mesothelin antibody according to any one of claims 27 to 40, wherein the VL CDR1, VL CDR2, and VL CDR3 sequences are present in a human VH framework.

42. The anti-mesothelin antibody according to any one of claims 27 to 41, wherein the light chain variable region has at least 95% sequence identity to SEQ ID NO: 52.

43. The anti-mesothelin antibody according to any one of claims 19 to 42, wherein the anti-mesothelin antibody specifically binds to human MSLN.

44. The anti-mesothelin antibody according to any one of claims 19 to 43, wherein the anti-mesothelin antibody is an isolated antibody.

45. An antibody fragment that specifically binds to mesothelin, wherein the antibody fragment comprises a fragment of the anti-mesothelin antibody according to any one of claims 19 to 44.

46. The antibody fragment according to claim 45, wherein the antibody fragment is a three-chain antibody like molecule.

47. A pharmaceutical composition comprising at least one mesothelin binding protein, antibody-drug conjugate, anti-mesothelin antibody, or antibody fragment according to any one of claims 13 to 46 and a pharmaceutically acceptable excipient.

48. A polynucleotide encoding the single domain antibody according to any one of claims 1 to 12.

49. A composition comprising one or more polynucleotide(s) encoding the mesothelin binding protein, anti-mesothelin antibody, or antibody fragment according to any one of claims 13 to 17 or 19 to 46.

50. A recombinant expression vector comprising the polynucleotide or composition according to claim 48 or 49.

51. A host cell comprising the recombinant expression vector according to claim 50.

52. A method of treating a disease associated with mesothelin expression in a subject in need thereof comprising administering to the subject at least one mesothelin binding protein, antibody-drug conjugate, anti-mesothelin antibody, or antibody fragment according to any one of claims 13 to 46.

53. The method according to claim 52, wherein the disease associated with mesothelin expression is chosen from mesothelioma, pancreatic cancer, gastric cancer, ovarian cancer, lung cancer, and triple negative breast cancer.
